(19) United States
(12) Patent Application Publication
(10) Pub. No.: US 2012/0309691 A1
(43) Pub. Date: Dec. 6, 2012

(54) TUMOR TARGETED DELIVERY OF IMMUNOMODULATORS BY NANOPOLYMERS

(76) Inventors: Dapeng Zhou, Houston, TX (US); Chun Li, Missouri City, TX (US); Patrick Hwu, Houston, TX (US)

(21) Appl. No.: 13/577,207

(22) PCT Filed: Feb. 3, 2011
(86) PCT No.: PCT/US11/23609

§ 371 Date: Aug. 3, 2012

Related U.S. Application Data
(60) Provisional application No. 61/301,252, filed on Feb. 4, 2010.

(51) Int. Cl. A61K 38/02; A61P 35/00; C07K 14/00

(52) U.S. Cl. 514/19.3; 530/358

(57) ABSTRACT

Nanoconstructs having three components: (1) biodegradable nanopolymers and nanoparticles, (2) immunodrugs such as CpG, and a (3) tumor binding device, which are actively targeted to tumor cells such as melanoma cells through receptor-mediated uptake and methods of using the same are described. Antitumor immunity is further enhanced by combination of PG-CpG nanoconstructs with agonists of positive costimulatory signals and inhibitors of negative immune regulatory signals.

### CROSS-REFERENCE TO RELATED APPLICATIONS

This application claims priority to U.S. Provisional Application Ser. No. 61/301,252 filed on Feb. 4, 2009. The application is incorporated by reference herein in its entirety.

### STATEMENT REGARDING FEDERALLY SPONSORED RESEARCH OR DEVELOPMENT

None.

### THE NAMES OF THE PARTIES TO A JOINT RESEARCH AGREEMENT

None.

### REFERENCE TO SEQUENCE LISTING

None.

### FIELD OF THE INVENTION

Nanoconstructs comprising biodegradable polymers conjugated to tumor binding ligands and antibodies which can target tumors with enhanced retention in tumor sites are described. Antitumor immunity is further enhanced by combination of the nanoconstructs with agonists of positive costimulatory signals and inhibitors of negative immune regulatory signals.

### BACKGROUND OF THE INVENTION

Melanoma is the deadliest of the skin cancers due to its propensity to widely metastasize throughout the body. Once it has spread to distal sites, the median survival is less than 6 months. Conventional therapies currently have limited efficacy against metastatic melanoma. There is now strong evidence that the immune system can play a significant role in inducing long-term benefits for some patients with metastatic melanoma. One approach towards the development of a strong immune response involves activation of innate immune cells such as plasmacytoid dendritic cells (“pDC”) by engaging specific toll like receptors (“TLRs”). TLR9 is the most specific of the human TLRs expression in pDCs and B cells that respond directly to stimulation by CpG oligodexoxynucleotide. Unfortunately, systemic injection of CpG causes activation of pDC cells in major immune organs, and exhausts the pool of this important type of anti-tumor cells outside of the tumor. A need exists therefore for targeted delivery of CpG to melanoma to enhance antitumor activity while reducing or eliminating systemic activation of pDC.

### SUMMARY OF THE INVENTION

Targeted delivery of CpG to melanoma in vivo through biodegradable L-PG is presented herein where this delivery system effectively generates the protective immunity required and enhances antitumor activity and reduces or even abolishes the systemic activation of pDC. Biodegradable polymers conjugated to tumor binding ligands and antibodies which can target tumors with enhanced retention in tumor sites are described. By way of example, one such polymer conjugate is poly(L-glutamic acid)-CpG conjugate (“L-PG-CpG”). L-PG-CpG has been shown to reduce tumor growth better than free CpG, and triggers a stronger systemic CD8 T response toward tumor antigen (OVA). This macrophage-tropic polymer interacts with tumor infiltrating macrophages and accumulates in tumor sites.

### BRIEF DESCRIPTION OF THE DRAWINGS

FIG. 1 provides two illustrations of intratumoral activation of pDC by TLR agonists primes tumor antigen-specific CD8 responses, which subsequently cause rejection of distal tumor. Specifically, FIG. 1A shows intratumoral injection of CpG induces a “priming phase” of immune response, as defined by the priming of antigen-specific adaptive immune cells (CD4 and CD8 T cells). In the priming phase, CpG activate pDC to produce INF-α, which further activates NK cells. NK cells lyse tumor cells and release tumor antigens to mDC. INF-α also activates mDC to become potent professional antigen-presenting cells. mDC subsequently migrate to tumor-draining lymph nodes, where they induce expansion of antigen-specific CD4 and CD8 T cells. FIG. 1B illustrates that after the CD4 and CD8 T cells are expanded in lymph nodes, they enter the blood circulation and trigger the “effector phase” of the adaptive immune response. In the effector phase, the antigen-specific CD8 T cells and other tumoricidal cells (NK cells) are recruited to tumor sites. Most importantly, CD8 T cells and NK cells can enter not only the primary tumors that receive intratumoral injection but also the distal metastatic tumors that are not accessible for intratumoral injection. Immune rejection of distal tumors is another major advantage of intratumoral CpG treatment.

FIG. 2 illustrates negative and positive costimulatory pathways that determine the tumoricidal activity of CD8 T cells. The tumoricidal activity of CD8 cells is regulated not only by recognition of tumor antigens through T cell receptor signaling pathway, but also by negative and positive costimulatory pathways. B7 negative costimulatory pathways, as represented by the CTLA4 pathway, turns off the tumoricidal activity of CD8 T cells. Positive costimulatory pathways such as OX40 and cytokines (IL-2) enhance the tumoricidal activity of CD8 cells. Antitumor reagents have been developed that target immune-costimulatory pathways. Antagonist antibodies against CTLA4 have shown initial promise in treatment of human renal cell carcinoma. Agonist antibody to OX40 has shown a significant increase in survival in tumor-bearing mice. Such reagents may be rationally combined with TLR9 agonists in cancer therapy.

FIG. 3 shows structures of PG and PG-based conjugates for magnetic resonance imaging (PG-Gd), near-infrared fluorescence imaging PG-NIR), dual optical/MR imaging (PG-Gd-NIR), and PG-CpG conjugates [PG-Gd(¹¹¹In)-CpG and PG-NIR-CpG], which are immunostimulatory agents visualizable with optical/MR imaging. PG is denoted as L-PG when the polyamino acid is composed of L-glutamic acid, and as D-PG when the polyamino acid is composed of D-glutamic acid, NIR, near-infrared. DTPA is diethylenetriaminpentaacetic acid.

FIG. 4 show only intratumorally injected CpG treats B16F10 melanoma in mice, B16F10 melanoma cells were subcutaneously inoculated in the right flank of C57BL6 mice. FLT3 ligand DNA (10 μg per mice) was injected into mice using a hydrodynamic method the same day as tumor inoculation to expand the dendritic cells in vivo. 7 days after tumor inoculation and dendritic cell expansion, 20 μg of CpG was injected. Control group means tumor-bearing mice with only FLT3 ligand treatment but no CpG treatment. CpG was delivered by intratumoral or intraperitoneal injection at a dose of 20 μg of CpG (in 50 μl of PBS) per injection.

FIGS. 5A, 5B & 5C show that PG-CpG enhances the immunostimulatory potency and antitumor efficacy of CpG. FIG. 5A shows that L-PG-CpG was more efficient than CpG against melanoma tumor transplants in mouse model, when administered intratumorally. Seven days after subcutaneous inoculation of B16-OVA tumor cells to C57B6 mice, L-PG-CpG (50 μg equivCpG), CpG (50 μg), or L-PG (500 μg) was injected into the tumor. Tumor size were measured every 3 days by measuring the perpendicular diameters of tumors (n=5). FIG. 5B shows that L-PG-CpG was more efficient than CpG in triggering antigen-specific immune responses, when administered intratumorally. Mice bearing subcutaneous B16-OVA tumors were treated as in A. Tumor antigen-specific CD8 responses were measured as OVA-specific CD8 T cell counts, using OVA257-264 peptide-loaded tetramers (BD Pharmingen, San Diego, Calif.). FIG. 5C shows L-PG-CpG, but not soluble CpG, specifically activated immune cells in the tumor. At 5 days after intratumoral injection of each drug, immune cells from tumor and spleen were extracted and analyzed for NK cell activation by staining with anti-CD69 antibody. PG-CpG retained its specificity for tumor. In contrast, soluble CpG activated NK cells in the spleen as well. Solid line: no-treatment control. Dashed line: treated with CpG or L-PG-CpG.

FIG. 6 illustrates the distribution of h-PG polymers after intratumoral injection. FIG. 6A is a NIRF image acquired at 24 h after injection of h-PG-NIR into human DM14 squamous cell carcinoma in the tongue of nude mice showed retention of the polymer at the injection site and the draining cervical lymph nodes (arrows). FIG. 6B is a NIRF image of resected tumor and lymph nodes. FIG. 6C is a microphotograph of an H&E-stained resected lymph node. FIG. 6D shows the retention of ¹¹¹In-labeled L-PG-CpG and CpG in B16 melanoma reviewed by autoradiography.

FIG. 7 is data obtained after inoculation with B16-OVA melanoma, mice were treated with PBS, CpG, anti-mouse OX40, or CpG plus anti-mouse OX40. FIG. 7A provides tumor area in mm³ over time. FIG. 7B provides the percentage of specific OVA antigen-positive CD8+ T cells out of total CD8 T cells over time.

FIG. 8 is the biodistribution of CpG and PG-CpG after intravenous injection. Significantly less PG-CpG was taken up by the liver and the spleen.

FIG. 9 shows PG-Gd-NIR was taken up by macrophages/APC in tumors. FIG. 9A shows PG-Gd-NIR co-localized with CD68 macrophages/APC markers in C6 tumors in nude rats 24 h after intravenous injection. FIG. 9B-D shows the depletion of macrophages/APC with clodronate liposomesin syngeneic Balb/c mice bearing A20 B-cell lymphoma led to reduced uptake of PG-Gd-NIR in tumors. Clodronate liposomes were injected 24 h prior to the injection of PG-Gd-NIR (0.02 mmol eq. Gd/kg, 48 nmol NIR dye per mouse). FIG. 9B provide near-infrared fluorescence images acquired with the FMT2500 3D optical imaging system. FIG. 9C is a T1-weighted MR images obtained at 4.7 T 2 days after injection of Pg-Gd-NIR. FIG. 9D is the immune-histological staining of exercised tumors confirming depletion of CD68+ cells and significant reduction of fluorescence intensity and the MRI signal in the tumors of mice injected with clodronate liposomes compared to mice injected with saline control.

FIG. 10 shows MSH-PG-CpG is metabolized by B16-F10 cells expressing MSH receptor, MSH-L-PG-CpG was incubated with B16-F10 cells in 6 well plates at a concentration of 200 μg/ml. The B16-F10 cells were allowed to take up MSH-PG-CpG for 2 h. Then the cells were washed 3 times with RPMI culture medium. Fresh culture medium was added, and the cells were cultured for 12 h, to allow the processing of the internalized MSH-PG-CpG. The culture medium was harvested, and the CpG released to culture medium was quantified by their stimulatory activity for production of IFN-γ by mouse splenocytes. The IFN-γ production was determined by ELISA assay with a kit from Pharmingen (San Diego, Calif.).

FIG. 11 shows structures of monoglutamate L-Glu-CpG, Gd(¹¹¹In)-, or fluorescent dye-labeled L-PG-CpG (4 different MWs), D-PG-CpG, poly(L-Glu-Tyr)-CpG, poly(L-Glu-Ala)-CpG, poly(hydroxypropyl L-glutamate)-CpG (L-PHPG-CpG), and L-PG-ketal-CpG and D-PG-ketal-CpG with acid-labile linkers.

FIG. 12 is a synthetic scheme for Gd-, ¹¹¹In-, or dye-labeled L-PHPG-CpG.

FIG. 13 is the synthesis of Gd (¹¹¹In) or dye-labeled NDP-MSH-PEG-L-PG-CpG (polymer 5) for targeted delivery of CpG.

FIG. 14A depicts the oxidation of tyrosine by tyrosinase to L-DOPA to orthoquinone. FIG. 14B is the synthesis of tyrosinase-activatable, CpG-bound DNP-MSH-PEG-D-PG nanoconstruct targeted to MC1R. In the presence of tyrosinase, the polymeric conjugate undergoes a Michael-type cyclization to release free CpG. FIG. 14C shows CpG is coupled to isocyanate derived from Tyr through a urea linker.

FIG. 15 shows PG-CpG-NIR was associated with macrophages (CD68+) in B16/F10 melanoma at 4 h after intratumoral injection. PG-CpG-NIR and tumor associated macrophages were highly distributed throughout the tumor. Macrophages engulfed the polymer into vesicular compartment (arrows) are shown at higher magnification. The polymer was probably phagocytized by the macrophages in the endo-lysosomal compartment.

FIG. 16 is an illustration of tumor targeting polymer-drug conjugates.

FIG. 17 is yet another illustration of the hypothesized mechanism for nanopolymer-CpG delivery.

FIG. 18 shows the purity of the nanopolymer CpG.

FIGS. 19A & B show PG-CpG activate splenic NK cells in vitro.

FIGS. 20A, B, C, & D show the selective uptake of PG polymer by tumor associated macrophages (CD11b+).

FIG. 21 shows the selective uptake of PG polymer by macrophages and DCs in draining lymph nodes, but not B cells.

### DETAIL DESCRIPTION OF THE INVENTION

Immunotherapeutics convert the immune-suppressive microenvironment to immune-stimulatory. Drugs acting on the innate arm of immune system have shown great promise due to their unique feature in “jump-starting” the immune responses. In the last decade, molecular identification of the receptors of the innate immune cells has led to discoveries and designs of a series of immunomodulators. Novel nanotechnology platforms and delivery systems are provided herein for the generation of an antitumor immune response through activation of plasmacytoid dendritic cells (pDC) using the Toll-like receptors (TLRs) TLR agonists that stimulate TLR9 signaling in immune cells.

Targeted delivery of a TLR9 agonist CpG to melanoma in vivo through biodegradable polymers effectively generating protective immunity and enhancing antitumor activity (while reducing or even abolishing the systemic activation of pDC) is described herein. Activation of pDC in major immune organs such as liver and spleen can exhaust the pool of this important type of antitumor cells outside of the tumor. Targeted delivery of a TLR9 agonist CpG to melanoma in vivo through biodegradable polymers effectively generating protective immunity and enhancing antitumor activity reduces or even abolishes the systemic activation of pDC. The described technology herein can also be useful in connection with the targeted delivery of the following: TLR1/2 agonist such as Pam3CSK4; TLR3 agonist such as poly(I:C); TLR4 agonist such as synthetic lipid A mimetics; TLR5 agonist such as flagellin; TLR6/2 agonist such as FSL-1 (Pam2CGDPKHPKSF); TLR7 agonist such as Imiquimod; TLR8 agonist such as ssRNA40; and NOD1/2 agonist such as Tri-DAP and muramyl dipeptide (MDP)

As provided herein, PG-CpG nanoconstructs actively targeted to melanoma cells through receptor-mediated uptake were developed. Antitumor immunity is enhanced by rational combination of PG-CpG nanoconstructs with agonists of positive costimulatory signals and inhibitors of negative immune regulatory signals.

Specifically, we have applied this macrophage-tropic polymer technology to deliver CpG ODN221.6 to tumor sites in a mouse model of melanoma. We synthesized poly(L-glutamic acid)-CpG conjugate (“L-PG-CpG”), and examined its anticancer effect as compared to non-conjugated CpG ODN2216 when administered intratumorally to B16-OVA melanoma subcutaneous transplant. We found that L-PG-CpG reduced tumor growth more than free CpG did. Furthermore, L-PG-CpG triggered a stronger systemic CD8 T cell response toward tumor antigen, OVA.

To further exploit the applications of this invention in immunotherapy of melanoma and the role of intratumoral injection of nano-CpG on the antitumor immunoresponse of CpG, a structure-activity relationship between the physicochemical characteristics of polymeric carriers and the immunostimulatory activity of CpG after intratumoral injection can be established. Nano-CpG targeted to melanoma cells (where the nano-CpG are processed locally by melanoma cells) can activate pDC in a tumor-specific manner. Also, building upon the fact that immune system is complex, the immunotherapy of melanoma described herein may require a combined interference on multiple immunostimulatory pathways, the combination of nano-CpG with other agonists of positive costimulatory pathways, and/or antagonists of negative costimulatory pathways.

Furthermore, melanoma is one of several solid tumors sensitive to immunotherapy. Other types of immunotherapy that have shown successes in treating melanoma patients, include high dose cytokines such as interferon-α (“TFN-α”) and interleukin 2 (“IL-2”), melanoma tumor antigen-based peptide vaccines, dendritic cell vaccines, and adoptively transferred tumor antigen-specific CD8 T cells. The mechanism that can lead to the response of melanoma to immunotherapy is the conversion of an immunosuppressive tumor microenvironment to an immune-stimulating tumor microenvironment. Immune activators, such as Toll-like receptor (“TLR”) agonist CpG oligonucleotides containing unmethylated cytosine-guanine motifs (generally referred to herein as “CpG”) significantly improve the efficacy of immunotherapy as shown in mouse models of melanoma.

Synthetic CpG mimic microbial DNA and elicit a coordinated set of immune responses, including innate and acquired immunity. Plasmacytoid dendritic cells (“pDC”) are a primary target cell of CpG in humans. pDC have an exceptional capacity to produce TEN-α, which subsequently activates T cells, natural killer (NK) cells, and other components of antitumor immunity. As shown in mouse models, CpG is an efficient immune modulator of cancer and has been proven to be safe in human clinical trials. Tumor site-specific delivery of free CpG, as systemic injection of CpG, however, causes activation of pDC in major immune organs such as liver and spleen and exhausts the pool of this important type of antitumor cells outside of the tumor. Intratumoral injection of CpG significantly enhances its antitumor effect, through “focusing” the immune stimulation in tumor sites. However, two problems exist for intratumoral injection of soluble CpG. First, it is difficult to control the retention time of injected CpG in the tumor. Second, soluble CpG can still be absorbed to circulation through diffusion. To overcome these problems, we propose the use of a biocompatible, biodegradable polymer platform to direct and control the release of CpG at the tumor sites.

Towards this aim and as we show herein, synthetic poly(L-glutamic acid) (“L-PG”) and other polymers can be selectively retained in tumors through phagocytosis by tumor-associated macrophages, providing a viable drug delivery system. Suitable polymers useful in connection with this invention include but are not limited to poly(DL-glutamic acid); poly(L-aspartic acid); poly(hydroxylpropyl glutamate; poly(hydroxylethyl glutamate); copolymers of poly(amino acids); and other synthetic and natural water-soluble polymers including but not limited to: polyvinyl alcohol, polyhydroxy ethyl methacrylamide, dextran, polysaccharides, human serum albumin, hyaluronic acid, and the like.

We found that the L-PG-CpG conjugate, that is, CpG chemically bound to L-PG delivered by intratumoral injection displays significantly greater antitumor activity against established melanoma tumors than did free CpG delivered by intratumoral injection. As further provided herein, the optimal physicochemical characteristics of PG-CpG to their anticancer effect following intratumoral injection can be determined by synthesizing and characterizing a battery of CpG-bound PG polymers (also referred to herein sometimes as “nano-CpG”) having different molecular weight (and thus size), degradability, and charge. The ability of the nanoconstructs to induce innate and acquired immunity after intratumoral injection can then be evaluated.

Furthermore, as described below, PG-CpG nanoconstructs actively targeted to melanoma cells through both receptor-mediated uptake and tyrosinase-mediated CpG release have been developed and validated. As yet further described herein, antitumor immunity can be enhanced by combination of PG-CpG nanocontructs with positive and negative costimulatory molecules. For example, the antitumor effect of combinations of nano-CpG and either agonist antibodies for positive costimulatory molecules (such as OX40), or antagonist antibodies for negative costimulatory molecules (such as CTLA-4 and B7) are proposed. Methods are provided for determining the antitumor effect of combinations of nano-CpG and therapeutic antibodies which act on costimulatory pathways in conjunction with cytokine regimens.

Vaccines based on the novel nano-CpG described herein can induce effective T-cell immune responses against melanoma using whole tumor as antigen. By utilizing these novel nanoconstructs for targeted delivery of immunostimulatory agents, improved antitumor efficacy can be produced. Furthermore, although melanoma has been demonstrated to be an excellent model system for testing immune strategies, the strategies described herein are applicable to treat other types of cancers, such as lung cancer and colon cancer.

### Immunotherapy for Melanoma.

In 2009, approximately 69,000 men and women in the United States were diagnosed with melanoma, and it was estimated approximately 8,600 will die from the disease. Jemal A. et al. Cancer Statistics, CA Cancer J Clin 59:225-49, 2009. Significantly, melanoma is being diagnosed with increasing frequency, and the incidence is increasing 3% per year. Melanoma is characterized by its high capacity for invasion and metastasis. Among patients with melanoma, approximately 20% eventually die of metastatic disease. Thus, melanoma remains one of the most common causes of death from malignancy. Once melanoma has spread to distant sites, the median survival is less than 6 months.

Over two decades ago, it was discovered that melanoma patients can mount a T-cell response against their tumor. Boon T, et al., Human T Cell Responses Against Melanoma, Annu Rev Immunot 24:175-208, 2006. Several immunologic therapies have been tested in melanoma patients, including interferon therapy, allogenic whole-cell vaccines, recombinant viral vectors, adoptive immunotherapy combined with lympho depletion, and allogenic cell lysates. There is now strong evidence that the immune system can play a significant role in inducing long-term benefits for some patients with metastatic melanoma. Overall response rates remain low, however, likely because of lack of melanoma-specific antitumor immune response and deficiency of strategies that only activate single steps in a complex immune response. Full activation of immunity requires stimulation of positive costimulatory signals and inhibition of negative immune regulatory signals. Emerging nanotechnology and the novel approaches described herein allow for stimulation of a positive immune response while reversing the immune-suppressive microenvironment. Intratumoral injection of immune activators such as TLR9 agonist CpG can enhance the efficacy of CD8(+) killing in a mouse model of melanoma.

### Activation of Innate Immunity is Critical for the Generation of Effective Adaptive Immune Responses.

The critical steps involved in the development of a strong immune response include activation of innate immune cells such as pDC by engaging specific TLR. Degli-Esposti M A, Smyth M J, Close Encounters of Different Kinds: Dendritic Cells and NK Cells Take Centre Stage. Nat Rev Immunol 5:112-24, 2005; Kadowaki N, Liu Y J, Natural Type I Interferon-Producing Cells as a Link Between Innate and Adaptive immunity, Hum Immunol 63:1126-32, 2002; Krutzik S R, et al., TLR Activation Triggers the Rapid Differentiation of Monocytes into Macrophages and Dendritic Cells, Nat Med 11:653-60, 2005. This in turn leads to activation of NK cells and myeloid dendritic cells (mDC), antigen release following lysis of target cells, and, finally, activation of specific Tcells (adaptive immunity). Activation of innate immunity induces the production of proinflammatory cytokines, which can directly activate cells important for the initiation of adaptive immune responses.

Type I IFNs, represented by IFN-α and IFN-β, and tumor necrosis factor (TNF-β), for example, are potent inducers of mDC maturation, inducing upregulation of major histocompatibility complex (MHC) and costimulatory molecules as well as production of IL-12, both of which are important for the priming of naïve T cells. Banchereau J, Steinman R M. Dendritic Cells and The Control of Immunity, Nature 392:245-52, 1998; Montoya M, et al. Type I interferons Produced By Dendritic Cells Promote Their Phenotypic and Functional Activation, Blood 99:3263-71, 2002.

In addition, activation of NK cells by pDC, cytokines, and TLR agonists may lead to increased lysis of tumors, which, in turn, can provide antigen to mDC for presentation to T cells. Activation of innate immunity is important not only for the generation of antigen-specific Teens, but also to induce inflammation, which leads to enhanced migration of antigen-specific Tcells to the tumor site.

### pDC Represent a Critical Link Between Innate and Adaptive Immunity.

As the major producer of type I IFNs, pDC represent one of the most important links between innate and adaptive immunity. Apostolou I, et al., Origin of Regulatory T Cells With Known Specificity For Antigen, Nat Immunol 3:756-63, 2002; Bjorck P., The Multifaceted Murine Plasmacytoid Dendritic Cell, Hum Immunol 63:1094-102, 2002; Gilliet M, et al., The Development of Murine Plasmacytoid Dendritic Cell Precursors is Differentially Regulated by FLT3-Ligand and Granulocyte/Macrophage Colony-Stimulating Factor, J Exp Med 195:953-8, 2002; Kadowaki N, et al., Subsets Of Human Dendritic Cell Precursors Express Different Toll-Like Receptors And Respond To Different Microbial Antigens, J Exp Med 194:863-9, 2001; Liu Y J., IPC: Professional Type I Interferon-Producing Cells and Plasmacytoid Dendritic Cell Precursors, Annu Rev Immunol 23:275-306, 2005.

Upon triggering of TLR7 or TLR9, pDC rapidly produce large amounts of type I IFNs, activate a variety of immune cells, such as B cells, NK cells, and macrophages, and differentiate into APC to induce antigen-specific T-cell responses. Nestle F O, et al., Plasmacytoid Predendritic Cells Initiate Psoriasis Through Interferon-Alpha Production, J Exp Med 202:135-43, 2005. Both mDC and NK cell activation can also be partially mediated by type I IFNs. IFN-α Rc−/−mDC are defective in the ability to adequately respond to viral infections, suggesting that IFN-producing pDC may be critical for the activation of mDC and subsequent development of adaptive immunity. Honda K, et al., Spatiotemporal Regulation of Myd88-IRF-7 Signaling For Robust Type-I Interferon Induction, Nature 434:1035-40, 2005.

A number of lines of evidence suggest that pDC may interact with mDC to induce an enhanced adaptive immune response in the development of antiviral immunity. Dalod M, et al. Dendritic Cell Responses To Early Murine Cytomegalovirus Infection: Subset Functional Specialization and Differential Regulation By Interferon Alpha/Beta, J Exp Med 197:885-98, 2003; Fonteneau J F, et al., Human Immunodeficiency Virus Type 1 Activates Plasmacytoid Dendritic Cells and Concomitantly Induces the Bystander Maturation Of Myeloid Dendritic Cells, J Virol 78:5223-32, 2004; Teleshova N, et al., Cpg-C Immunostimulatory Oligodeoxyribonucleotide Activation of Plasmacytoid Dendritic Cells In Rhesus Macaques to Augment The Activation of IFN-Gamma-Secreting Simian Immunodeficiency Virus-Specific T Cells, J Immunol 173:1647-57, 2004. Activation of mDC by double-stranded RNA or viral infection has been shown to be dependent on exposure to IFN-α. Honda K, et al., Selective Contribution of IFN-Alpha/Beta Signaling To The Maturation of Dendritic Cells Induced By Double-Stranded RNA or Viral Infection, Proc Natl Acad Sci USA 100:10872-7, 2003; Radvanyi L G, et al., Low Levels of Interferon-Alpha Induce CD86 (B7.2) Expression and Accelerates Dendritic Cell Maturation From Human Peripheral Blood Mononuclear Cells, Scand J Immunol 50:499-509, 1999; Tough D F., Type I Interferon as A Link Between Innate and Adaptive Immunity Through Dendritic Cell Stimulation, Leuk Lymphoma 45:257-64, 2004. In addition, HIV was found to be able to activate pDC, which could subsequently activate mDC upon co-culture. Fonteneau J F, al., Human Immunodeficiency Virus Type 1 Activates Plasmacytoid Dendritic Cells and Concomitantly Induces The Bystander Maturation Of Myeloid Dendritic Cells, J Virol 78:5223-32, 2004. In addition, it has recently been demonstrated that pDC may interact with lymph node mDC in the generation of anti-HSV CTL. Tough, D F., Type I interferon As a Link Between Innate and Adaptive Immunity Through Dendritic Cell Stimulation, Leuk Lymphoma 45:257-64, 2004. These observations indicate that pDC are “jump-starters” of the adaptive immune responses toward viral infection and cancer.

### TLR9 and CpG as an Immunostimulatory Agent.

The TLR family consists of 13 different receptors recognizing microbial DNA and RNA structures. TLR agonists have been found to play integral roles in the activation of pDC, mDC, B cells, and macrophages. TLR9 is the most specific of the human TLRs due to its selective expression in pDC and B cells that respond directly to CpG stimulation. Three classes of CpG TLR agonists have been identified so far. Phosphorothioate B-class CpG, such as CpG7909, stimulate B cells and NK cells but induce only moderate amounts of IFN-α from pDC.

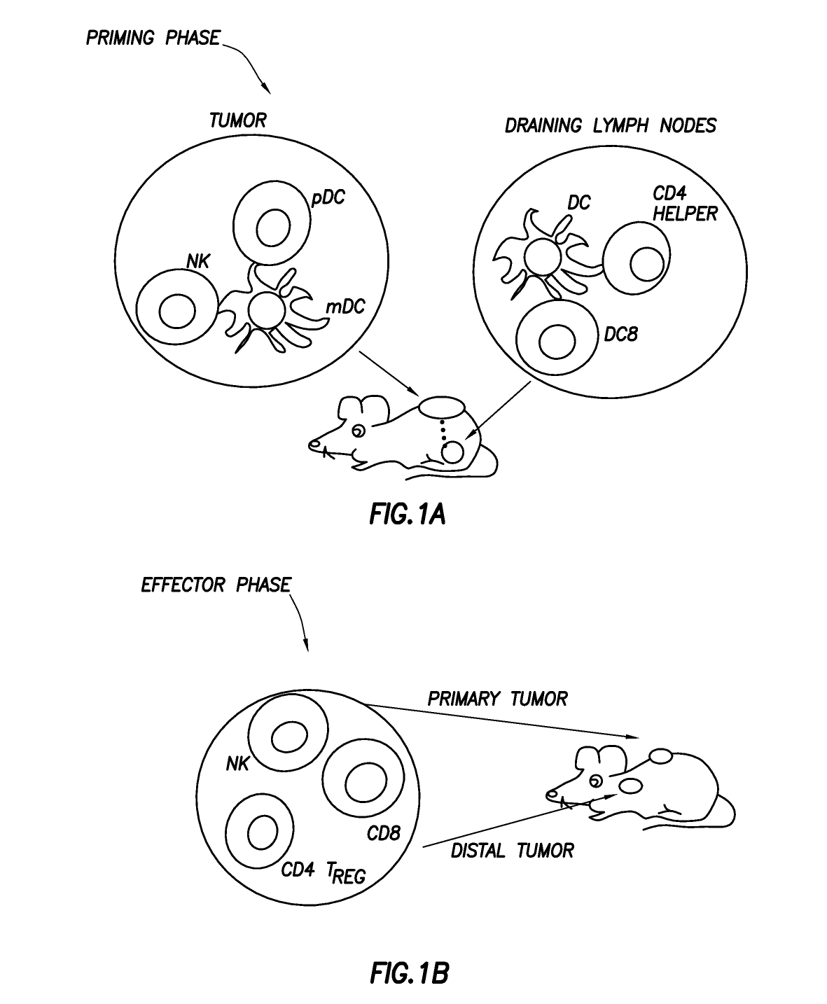

In contrast, A-class CpG, such as ODN2336 and ODN2216, induce extremely high amounts of type I IFN from pDC and high degrees of NK stimulation but have little B cell stimulatory capacity. ODN2216, an A-class CpG ligand activates pDC and NK cells in mouse and human, Vollmer J., Progress in Drug Development of Immunostimulatory CpG Oligodeoxynucleotide Ligands For TLR9, Expert Opin Biol Ther 5:673-82, 2005; Colonna, M., TLR Pathways and IFN-Regulatory Factors: To Each Its Own, Eur J Immunol 37:306-9, 2007. These cells subsequently activate MDC and induce tumor antigen-specific CD8 responses. See, FIG. 1. Significantly, we have demonstrated that intratumoral injection of CpG-activated pDC caused immune rejection of distal tumors. Liu C, et al., Plasmacytoid Dendritic Cells Induce NK Cell-Dependent, Tumor Antigen-Specific T Cell Cross-Priming and Tumor Regression in Mice, J Clin Invest 118:1165-75, 2008.

### Targeting Costimulatory Pathways.

Many melanomas remain refractory to immunotherapy despite large numbers of tumor-infiltrating CD8 T cells. One of the major mechanisms for the failure of immunotherapy is the immunosuppressive microenvironment within the tumor. Lizee G, et al., Improving Antitumor Immune Responses by Circumventing Immunoregulatory Cells and Mechanisms, Clin Cancer Res 12:4794-803, 2006: Liizee G, et al., Immunosuppression in Melanoma Immunotherapy: Potential Opportunities for Intervention, Clin Cancer Res 12:2359s-65s, 2006.

We have shown that (i) CD8 CTL are a major factor causing tumor regression and depletion of CD8 T cells significantly reduces the treatment effect of CpG; and (ii) CD8 T cells are the effector cell population for multiple immunomodulators, including anti-CTLA-4 antibody and anti-OX40 antibody. Liu C, Lou Y, Lizee G, et al., Plasmacytoid Dendritic Cells Induce NK Cell-Dependent, Tumor Antigen-Specific T Cell Cross-Priming and Tumor Regression In Mice, J Clin Invest 118:1165-75, 2008; Croft M, et al., The Significance of OX40 And OX40L to T-Cell Biology and Immune Disease, Immunol Rev 229:173-91, 2009; Redmond W L, Ruby C E, Weinberg A D, The Role Of OX40-Mediated Co-Stimulation in T-Cell Activation and Survival, Crit Rev Immunol 29:187-201, 2009.

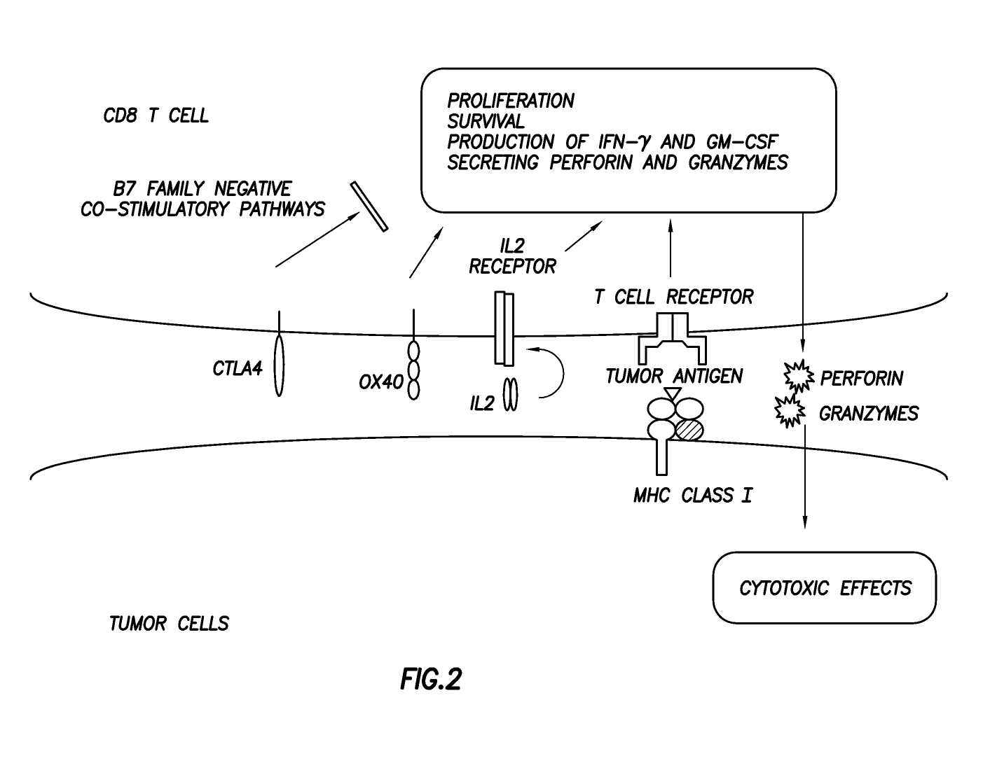

TNF family ligands define niches for T cell memory. Trends Immunol 28:333-9, 2007. These results form the basis (rationale) for combining TLR-agonists and reagents targeting immune costimulatory pathways. See, FIG. 2.

Blockade of negative costimulatory signals has been used for antitumor therapy. Clinical trials using blocking antibodies against CTLA-4, a molecule on T cells that dampens initial T-cell activation and proliferation, have had some success at activating the host immune response against melanoma. Montoya M, et al., Type I interferons Produced By Dendritic Cells Promote Their Phenotypic and Functional Activation, Blood 99:3263-71, 2002; Apostolou I, et al., Origin of Regulatory T Cells with Known Specificity For Antigen, Nat Immunol 3:756-63, 2002; Bjorck P., The Multifaceted Murine Plasmacytoid Dendritic Cell, Hum Immunol 63:1094-102, 2002. However, CTLA-4 blockade has profound effects on the extent of multiple T-cell responses, and autoimmunity is a major side effect. More targeted approaches inhibiting other negative costimulatory signals operating during and after T-cell activation, especially in tumor-infiltrating lymphocytes at the tumor site, is another approach to manipulating these negative signals for therapeutic purposes. The B7 family of molecules and its receptors expressed on cells are one of the “turn off” mechanisms that impede an effective immune response against tumors. Martin-Orozco N, Dong C., New Battlefields for Costimulation, J Exp Med 203:817-20, 2006; Martin-Orozco N, Dong C. Inhibitory Costimulation and Anti-Tumor Immunity, Semin Cancer Biol 17:288-98, 2007.

Recently several new B7 molecules, including B7S1, B7S3, and B7H3, and their function as negative regulators of T cells were described. Prasad, D V, et al., B7S1, A Novel B7 Family Member That Negatively Regulates T Cell Activation, Immunity 18:863-73, 2003; Sica G L, et al., B7-H4, a Molecule Of The B7 Family, Negatively Regulates T Cell Immunity, Immunity 18:849-61, 2003; Zang, X, et al. B7x: A Widely Expressed B7 Family Member That Inhibits T Cell Activation, Proc Natl Acad Sci USA 100:10388-92, 2003. These new molecules are broadly expressed in lymphoid and nonlymphoid tissues, in particular in APC. Some of these molecules have also been found up-regulated in tumors; for example, B7S1 is present in tumors originating from ovarian, breast, renal, and lung tissues. Prasad, D V, et al., B7S1, A Novel B7 Family Member That Negatively Regulates T Cell Activation, Immunity 18:863-73, 2003; Krambeck A E, et al., B7-H4 Expression in Renal Cell Carcinoma And Tumor Vasculature: Associations with Cancer Progression and Survival, Proc Natl Acad Sci USA 103:10391-6, 2006; Tringler B, et al., B7-H4 Overexpression in Ovarian Tumors, Gynecol Oncol 100:44-52, 2006; Tringler B, et al., B7-H4 is Highly Expressed in Ductal And Lobular Breast Cancer, Clin Cancer Res 11:1842-8, 2005. Blockade of these B7 molecules potently enhances T-cell proliferation and IL-2 production in vitro and increases autoreactive T cells in vivo. Prasad, D V, et al., B7S1, A Novel B7 Family Member That Negatively Regulates T Cell Activation, Immunity 18:863-73, 2003. Blocking B7S1 during T-cell vaccination in a mouse model of metastatic melanoma appears to substantially protect the mice from tumor development and that survivor mice are fully protected against a second tumor challenge (unpublished data). Targeting B7 molecules in synergy with TLR agonists can have tremendous therapeutic value in treating human melanoma.

Therefore, targeted activation of costimulatory molecules such as OX40, CD40, and 4-1BB constitutes another option to enhance T-cell activation to improve T-cell-mediated antitumor responses. Previously published studies of OX40 have revealed its importance in enhancing such T-cell effector functions as proliferation, cytokine production, and survival, and stimulation through OX40 has been found to be integral in the development of memory T-cells Croft, M., The Role of TNF Superfamily Members in T-Cell Function and Diseases, Nat Rev Immunol 9:271-85, 2009. Specific to tumor microenvironment, systemic administration of agonist anti-OX40 antibodies has been found to decrease the number of regulatory T cells, which function to suppress effector T cell activity, and increase the number of CD8+ T cells within tumors. Redmond W L, Ruby, C E, &. Weinberg, A D, The Role of OX40-Mediated Co-stimulation in T-cell Activation and Survival, Crit Rev Immunol 29:187-201, 2009. An agonist antibody to mouse OX40 used in mouse models of other tumor systems, such as sarcoma, colorectal carcinoma, and mammary carcinoma, has shown a significant increase in survival. Redmond, W L, et al., Ligation of the OX40 Costimulatoiy Receptor Reverses Self-Ag and Tumor-Induced CD8 T-Cell Anergy In Vivo, Eur J Immunol 39:2184-94, 2009; Song A, et al., OX40 and Bcl-xL Promote The Persistence Of CD8 T Cells To Recall Tumor-Associated Antigen, J Immunol 175:3534-41, 2005.

CD40 has previously been found to play a significant role in B cell activation, proliferation, and antigen presentation, as well as in dendritic cell activation and antigen presentation. Croft, M., The Role of TNF Superfamily Members in T-Cell Function and Diseases, Nat Rev Immunol 9:271-85, 2009. Agonist antibodies to CD40 have been found to overcome CD4+ T cell tolerance and enhance T cell cytotoxicity. Interestingly, CD40 is expressed by roughly 70% of solid tumor malignancies, including breast, colon, lung, and prostate cancers, and melanoma. Hurwitz A A, Kwon E D, van Elsas A., Costinmlatory Wars: The Tumor Menace, Curr Opin immunol 12:589-96, 2000. Agonist anti-CD40 antibodies have been evaluated in several murine models of cancer, but specific to melanoma, such antibodies were found only to slow tumor growth Melief, C J., Cancer immunotherapy by Dendritic Cells, Immunity 29:372-83, 2008. Phase I clinical trials are underway with agonist antibodies targeting multiple myeloma, non-Hodgkins lymphoma, melanoma, and chronic lymphocytic leukemia. Schaffner, E J., CD40 Ligand in CLL Pathogenesis and Therapy, Leuk Lymphoma 37:461-72, 2000.

4-1BB has been shown to enhance T cell cytokine production, proliferation, and cytotoxic activity, it may also play an integral role in establishing memory CTL. Agonist antibodies can eradicate established tumors in mouse models of sarcoma and mastocytoma. Lynch, D H., The Promise of 4-1BB (CD137)-Mediated Immunomodulation and the Immunotherapy of Cancer, Immunol Rev 222:277-86, 2008. Of interest, agonist anti-4-1BB antibodies may function to ameliorate autoimmune conditions and limit autoimmune side effects of immunotherapy in mice. Id. Although cancer treatments based on individual TLR agonist or antibody therapy have been well studied, the optimal strategy of combining TLR agonists and antibody therapy has not, despite great potential.

Lastly, the moderate clinical success seen with the administration of IL-2 and IFN-α to melanoma patients leaves room tbr improvement, potentially through the addition of a TLR-agonist, IFN-α was the first exogenous cytokine to demonstrate antitumor activity in advanced melanoma. In 1995, INF-2β, a different recombinant form of IFN-α, became the first FDA approved immunotherapy for adjuvant treatment of stageIIB/III melanoma. Kirkwood J M, et al., Next Generation of Immunotherapy for Melanoma, J Clin Oncol 26:3445-55, 2008. Studies showed that high-dose IFN-2β significantly reduced the risk of recurrence. Kirkwood J M, et al., Mechanisms and Management of Toxicities Associated with High-Dose Interferon alfa-2b Therapy, J Clin Oncol 20:3703-18, 2002. IL-2, the second exogenous cytokine to demonstrate antitumor activity against melanoma, was approved by FDA in 1998 for treatment of adults with advanced metastatic melanoma. Phan G Q, et al., Factors Associated with Response to High-Dose Interleukin-2 in Patients with Metastatic Melanoma, J Clin Oncol 19:3477-82, 2001. IL-2 plays a central role in immune regulation and T-cell proliferation. High-dose bolus intravenous IL-2 was shown to have antitumor effects in patients with advanced metastatic melanoma. Stoutenburg J P, Schrope B, & Kaufman, H L, Adjuvant Therapy for Malignant Melanoma, Expert Rev Anticancer Ther 4:823-35, 2004; Rosenberg S A, &. White, D E., Vitiligo in Patients with Melanoma: Normal Tissue Antigens can be Targets for Cancer Immunotherapy, J Immunother Emphasis Tumor Immunol 19:81-4, 1996. Retrospective long-term analysis of these phase II studies demonstrated an objective response rate of 16% with a durable response rate of 4%, suggesting that a memory T-cell response was established. Kirkwood J M, et al., Next Generation of Immunotherapy for Melanoma, J Clin Oncol 26:3445-55, 2008.

### Nanoparticles for Delivery of CpG.

A major obstacle to the clinical application of CpG as a potent and tumor-specific immunostimulatory agent is the need for an efficient delivery system. Free CpG as well as other stable phosphorothioate oligonucleotides administered by intravenous injection are cleared rapidly with a broad tissue distribution. Link B K, et al., Oligodeoxynucleotide CpG 7909 Delivered as Intravenous Infusion Demonstrates Immunologic Modulation in Patients With Previously Treated Non-Hodgkin Lymphoma, J Immunother 29:558-68, 2006; Wang H, et al., Immunomodulatory Oligonucleotides as Novel Therapy for Breast Cancer: Pharmacokinetics, In Vitro And In Vivo Anticancer Activity, and Potentiation Of Antibody Therapy, Mol Cancer Ther 5:2106-14, 2006: Yu R Z, et al., Comparison of Pharmacokinetics and Tissue Disposition of an Antisense Phosphorothioate Oligonucleotide Targeting Human Ha-Ras mRNA in Mouse and Monkey, J Pharm Sci 90:182-93, 2001. This is thought to contribute to the observed failure of systemically administered free CpG to elicit appreciable immune responsiveness in human volunteers and to the observed induction of anon-specific, generalized activation of the immune system that may be deleterious. Krieg A M, et al., Induction of Systemic TH1-Like Innate Immunity in Normal Volunteers Following Subcutaneous but not Intravenous Administration Of CPG 7909, A Synthetic B-Class CpG Oligodeoxynucleotide TLR9Agonist, J Immunother 27:460-71, 2004; Krieg A M. Therapeutic potential of Toll-like Receptor 9 Activation, Nat Rev Drug Discov 5:471-84, 2006.

Nanotechnology offers the potential for targeting CpG to APC, particularly to pDC in the tumor. Nanoparticles containing CpG generally exert better immunotherapeutic activity than free CpG following systemic administration, owning to the natural ability of APC to accumulate CpG nanoparticles and the depot effect, in which persistence of CpG at the site of action would provide enhanced activity, Whitmore M M, et al. Systemic Administration of LPD Prepared With Cpg Oligonucleotides Inhibits the Growth of Established Pulmonary Metastases By Stimulating Innate and Acquired Antitumor immune Responses, Cancer Immunol Immunother 50:503-14, 2001; Sakurai F, et al., Therapeutic Effect of Intravenous Delivery of Lipoplexes Containing The Interferon-[Beta] Gene And Poly I: Poly C In A Murine Lung Metastasis Model, Cancer Gene Ther 10:661-8; Higgins R, et al. Growth Inhibition Of An Orthotopic Glioblastoma In Immunocompetent Mice By Cationic Lipid-DNA Complexes, Cancer Immunol Immunother 53:338-44, 2004. Thus far, the subcutaneous route of administration has been tested and shown to result in significantly enhanced immunostimulatory and antitumor activities in animal models of melanoma with several CpG nanoparticles. de Jong S, et al., Encapsulation in Liposomal Nanoparticles Enhancer The Immunostimulatory, Adjuvant and Anti-Tumor Activity of Subcutaneously Administered CPG ODN, Cancer Immunol Immunother 56:1251-64, 2007; Standley S M, et al., Incorporation of CpG Oligonucleotide Ligand Into Protein-Loaded Particle Vaccines Promotes Antigen-Specific CD8 T-Cell Immunity, Bioconjug Chem 18:77-83, 2007; Li W M, Bally M B, Schutze-Redelmeier M P, Enhanced Immune Response To T-Independent Antigen By Using CpG Oligodeoxynucleotides Encapsulated in Liposomes, Vaccine 20:148-57, 2001; Bourquin C, et al., Targeting CpG Oligonucleotides to the Lymph Node by Nanoparticles Elicits Efficient Antitumoral Immunity, J Immunol 181:2990-8, 2008. However, subcutaneous CpG nanoparticles often require co-incorporation of tumor-associated antigens (TAA) into the CpG nanoparticles in order to induce tumor-specific CTL response. Intratumoral or peritumoral administration, on the other hand, may allow for the TLR9 “danger signal” to occur in the presence or close proximity of the TAA from the tumor itself.

In our preliminary studies, direct intratumoral injection of free CpG has shown promise in an animal model of melanoma. Moreover, intratumoral injection of a polymer-CpG conjugate has shown better antitumor activity than intratumoral injection of free CpG. These encouraging preliminary data, lead us to hypothesize that polymer-CpG conjugates delivered to tumors, where they are exposed directly to the specific tumor microenvironment and immune cell populations, may significantly enhance the antitumor immune response without activating systemic immunity.

Ultimately, what kills patient with melanoma is metastatic disease. Thus, in an ideal situation, CpG should be delivered systemically so that this TLR9 agonist has a chance to home to melanoma metastases. The challenge is to achieve local immune activation without inducing a systemic immune response. Nanotechnology offers a great opportunity to achieve this goal. To date, tumor-selective delivery of CpG nanoparticles has not been investigated. In this proposed work, we intend to formulate and evaluate water-soluble polymer-CpG targeted to melanoma to induce tumor-specific immune responses.

### L-PG as a Drug Carrier.

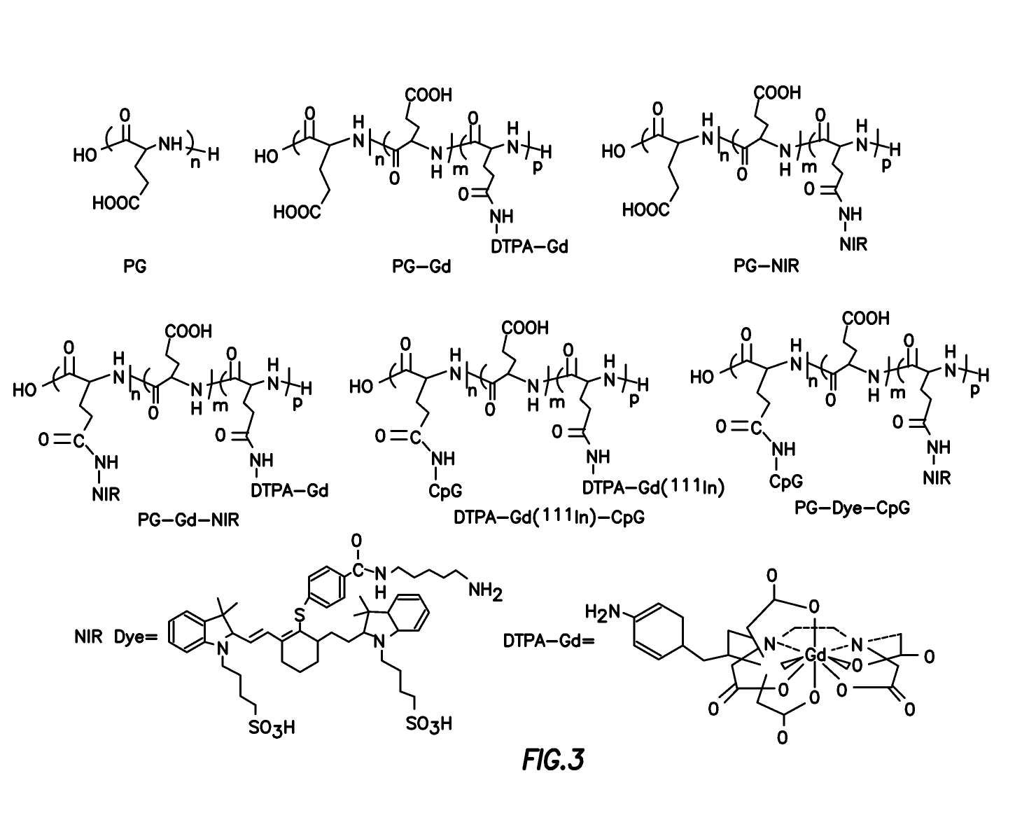

L-PG is unique in that it is composed of naturally occurring L-glutamic acid linked together through an amide bond backbone. The pendent free γ-carboxyl group in each repeating unit of L-glutamic acid is negatively charged at a neutral pH, which renders the polymer water-soluble. The carboxyl groups also provide functionality for attachment of multiple components, including drug molecules and imaging agents. See, FIG. 3. For example, an L-PG-paclitaxel conjugate developed in our laboratory, in which paclitaxel is covalently linked at the 2′-hydroxyl group by an ester bond to L-PG, has shown significant antitumor activity in a variety of preclinical animal tumor models and in early phase I trials. Li C, et al., Biodistribution of Paclitaxel and Poly(L-glutamic acid)-paclitaxel Conjugate in Mice with Ovarian OCa-1 Tumor, Cancer Chemother Pharmacol 46:416-22, 2000; Li C, et al., Antitumor Activity of Poly(L-glutamic acid)-paclitaxel on Syngeneic and Xenografted Tumors, Clin Cancer Res 5:891-7, 1999; Li C, et al., Complete Regression of Well-Established Tumors Using a Novel Water-soluble poly(L-glutamic acid)-paclitaxel Conjugate, Cancer Res 58:2404-9, 1998; Boddy A V, et al. A Phase I and Pharmacokinetic Study of Paclitaxel Poliglumex (XYOTAX), Investigating Both 3-Weekly and 2-Weekly Schedules, Clin Cancer Res 11:7834-40, 2005.

L-PG-paclitaxel is degraded into both mono- and di-glutamyl paclitaxel in vitro by macrophage-like cells and in vivo by a variety of tumors. Shaffer S A, et al., In Vitro and In Vivo Metabolism of Paclitaxel Poliglumex: Identification of Metabolites and Active Proteases, Cancer Chemother Pharmacol 59:537-48, 2007. The cysteine protease cathepsin B is an important mediator of L-PG degradation in tumors, although other proteolytic pathways contribute as well Shaffer S A, et al., In Vitro and In Vivo Metabolism of Paclitaxel Poliglumex: Identification of Metabolites and Active Proteases, Cancer Chemother Pharmacol 59:537-48, 2007; Melancon M P, et al., A Novel Method for Imaging In Vivo Degradation of Poly(L-Glutamic Acid), a Biodegradable Drug Carrier, Pharm Res 24:1217-24, 2007. This L-PG based anticancer agent is the first synthetic polymeric drug that has advanced to clinical phase III studies Li C, Wallace S., Polymer-Drug Conjugates: Recent Development In Clinical Oncology, Adv Drug Deli); Rev 60:886-98, 2008. L-PG is water-soluble, biodegradable, and nontoxic. A versatile chemistry is available for the synthesis of PG-based polymers with well-controlled molecular weight, degradability, and charge. These features make L-PG a particularly promising candidate as a carrier of CpG, and for understanding the structure-activity relationship between polymeric carriers and immunostimulatory activity of CpG.

### Melanocortin Type 1 Receptors and Tyrosinase as Therapeutic Targets of Melanoma.

Melanocortin type 1 receptor (MC1R) is overexpressed in melanoma cells. Giblin M F, et al., Design and Characterization of Alpha-Melanotropin Peptide Analogs Cyclized Through Rhenium and Technetium Metal Coordination, Proc Natl Acad Sci USA 95:12814-8, 1998; Miao Y, Benwell K, Quinn T P., 99mTc- and 111In-labeled Alpha-Melanocyte-stimulating Hormone Peptides as Imaging Probes for Primary and Pulmonary Metastatic Melanoma Detection, J Nucl Med 48:73-80, 2007; Lopez M N, et al., Melanocortin 1 Receptor is Expressed by Uveal Malignant Melanoma and can be Considered a New Target for Diagnosis and Immunotherapy, Invest Ophthalmol Vis Sci 48:1219-27, 2007.

[Nle⁴,D-Phe⁷]α-melanocyte-stimulating hormone (“NDP-MSH”) a small-molecular-weight peptide, is a potent agonist of MC1R that binds to MC1R with high affinity (IC₅₀=0.21 nM). Chen J, et al., Melanoma-Targeting Properties of (99m)Technetium-Labeled Cyclic Alpha-Melanocyte-Stimulating Hormone Peptide Analogues, Cancer Res 60:5649-58, 2000; Sawyer T K, et al., 4-Norleucine, 7-D-Phenylalanine-Alpha-Melanocyte-Stimulating Hormone: A Highly Potent Alpha-Melanotropin With Ultralong Biological Activity, Proc Natl Acad Sci USA 77:5754-8, 1980. NDP-MSH and other α-MSH analogues have been proposed as melanoma-preventative agents that work by preventing malignant transformation from melanocytes to melanoma. Abdel-Malek Z A., et al., The Melanocortin 1 Receptor and The UV Response of Human Melanocytes—A Shift In Paradigm, Photochem Photobiol 84:501-8, 2008. We have recently conjugated NDP-MSH to hollow gold nanospheres (˜40 nm in diameter) and have demonstrated MC1R-mediated active targeting of gold nanoparticles to B16 melanoma after intravenous injection using both optical and positron emission tomography imaging techniques. Lu W, Xiong C, Zhang G, et al., Targeted Photothermal Ablation of Murine Melanomas with Melanocyte-Stimulating Hormone Analog-Conjugated Hollow Gold Nanospheres, Clin Cancer Res 15:876-86, 2009. About 12.6% of injected dose was delivered to B16 melanoma after intravenous injection, which was significantly more than the dose delivered to the spleen (4%). These data suggest that NDP-MSH is an excellent homing ligand for targeted delivery of nanoparticles to melanoma.

Targeting nanoparticles to tumor-associated receptors, although attractive, cannot completely avoid nanoparticle uptake by the phagocytic cells in the liver and the spleen. In an ideal case, the CpG nanoparticles would release CpG solely at the tumor site upon the action of tumor-specific enzyme. In this way, the CpG-induced activation of immune effector cells at organs other than the tumor would be greatly reduced even though some of the injection nanoparticles are distributed to these organs (i.e. liver and spleen). This approach has already been exploited in cancer chemotherapy in the antibody-directed enzyme prodrug therapy (“ADEPT”) protocol. Jungheim L, Shepherd T., Design of Antitumor Prodrugs: Substrates for Antibody Targeted Enzymes, Chem Rev 94:1553-66, 1994. However, the ADEPT approach has its own limitation as the antibody-enzyme conjugate can bind to other tissues nonspecifically. Fortunately, it is possible to rely upon the enzyme tyrosinase, which is already present in melanoma cells and is uniquely associated with melanocytes. When melanocytes become malignant, the gene expressing tyrosinase become up-regulated, resulting in a marked increase in the tyrosinase levels within the cancer cells. Land E J, Ramsden C A, Riley P A, Quinone Chemistry and Melanogenesis, Methods Enzymol 378:88-109, 2004; Riley P A., Melanogenesis and Melanoma, Pigment Cell Res 16:548-52, 2003. Thus, since tyrosinase is naturally present in the tumor and virtually absent from other cells, it provides a built-in drug targeting mechanism. Alena F, Jimbow K, Ito S., Melanocytotoxicity and Antimelanoma Effects of Phenolic Amine Compounds in Mice In Vivo, Cancer Res 50:3743-7, 1990; Jordan A M, et al., Melanocyte-Directed Enzyme Prodrug Therapy (MDEPT): Development of Second Generation Prodrugs For Targeted Treatment of Malignant Melanoma, Bioorg Med Chem 9:1549-58, 2001; Jordan A M, et al., Melanocyte-directed Enzyme Prodrug Therapy (MDEPT): Development of a Targeted Treatment for Malignant Melanoma, Bioorg Med Chem 7:1775-80, 1999; Knaggs S, et al., New Prodrugs Derived from 6-aminodopamine and 4-aminophenol as Candidates for Melanocyte-Directed Enzyme Prodrug Therapy (MDEPT), Org Biomol Chem 3:4002-10, 2005; Morrison M E, Yagi M J, Cohen G., In Vitro Studies of 2,4-Dihydroxyphenylalanine, A Prodrug Targeted Against Malignant Melanoma Cells, Proc Natl Acad Sci USA 82:2960-4, 1985. A number of tyrosinase-dependent prodrugs have been tested for the treatment of melanoma.

For example, tyrosinase is utilized to mediate the release of cytotoxic agents from carbamate and urea prodrugs via a cyclization-drug release mechanism. Jordan A M, et al., Melanocyte-Directed Enzyme Prodrug Therapy (MDEPT): Development of Second Generation Prodrugs For Targeted Treatment of Malignant Melanoma, Bioorg Med Chem 9:1549-58, 2001; Jordan A M, et al. Melanocyte-Directed Enzyme Prodrug Therapy (MDEPT): Development of Targeted Treatment Malignant Melanoma, Bioorg Med Chem 7:1775-80, 1999. However, such mechanism has not been utilized for the delivery of immunostimulatory agents,

### Preliminary Results

Intratumoral Injection of CpG is Better than Systemic Injection.

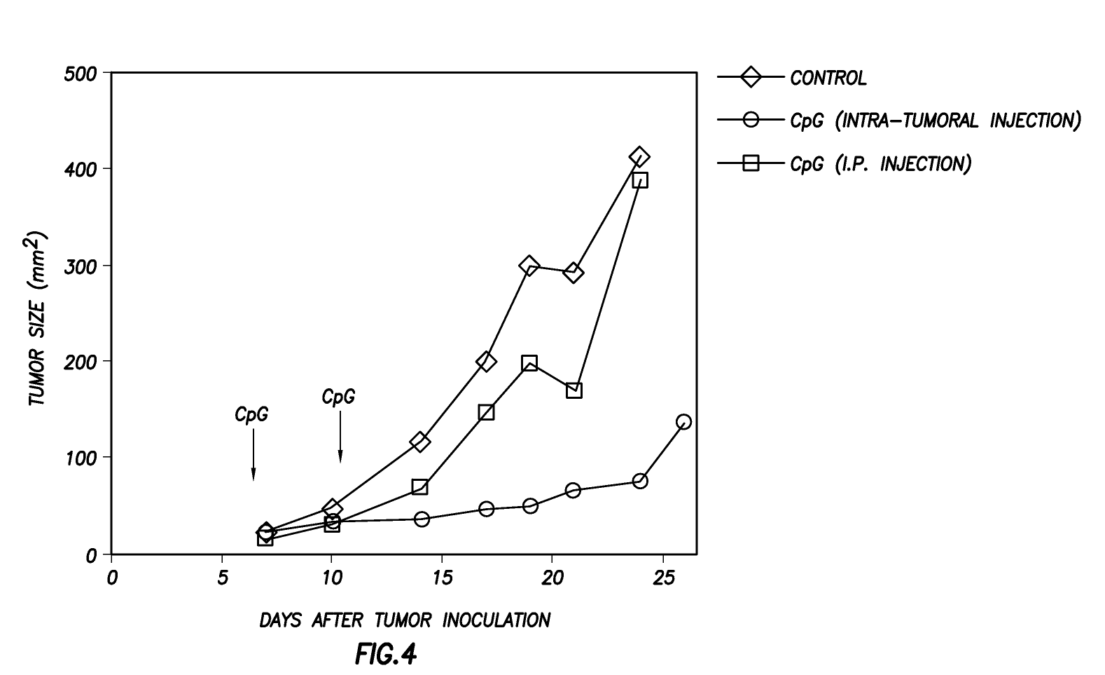

As shown in FIG. 4, we found that intratumoral injection of CpG significantly reduced B16F10 tumor growth, while this effect was not seen with intraperitoneal injection of CpG. We also found that the route of CpG in combination with cancer vaccine was critical. Although intravenous injection of CpG was able to induce activation and expansion of tumor antigen-specific T-cell response, most activated T cells failed to migrate to tumor. By contrast, intratumoral injection of CpG led to extensive tumor infiltration by antigen-specific T cells. These results led us to conclude that CpG acts locally and must be concentrated at the tumor site.

### L-PG-CpG Enhances the Immunostimulatory Potency and Antitumor Efficacy of CpG.

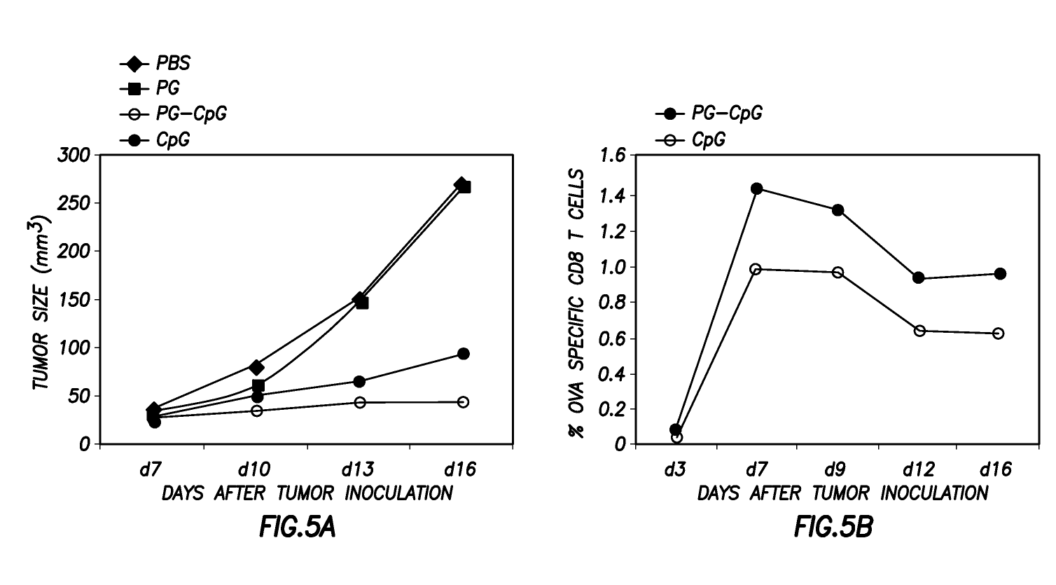

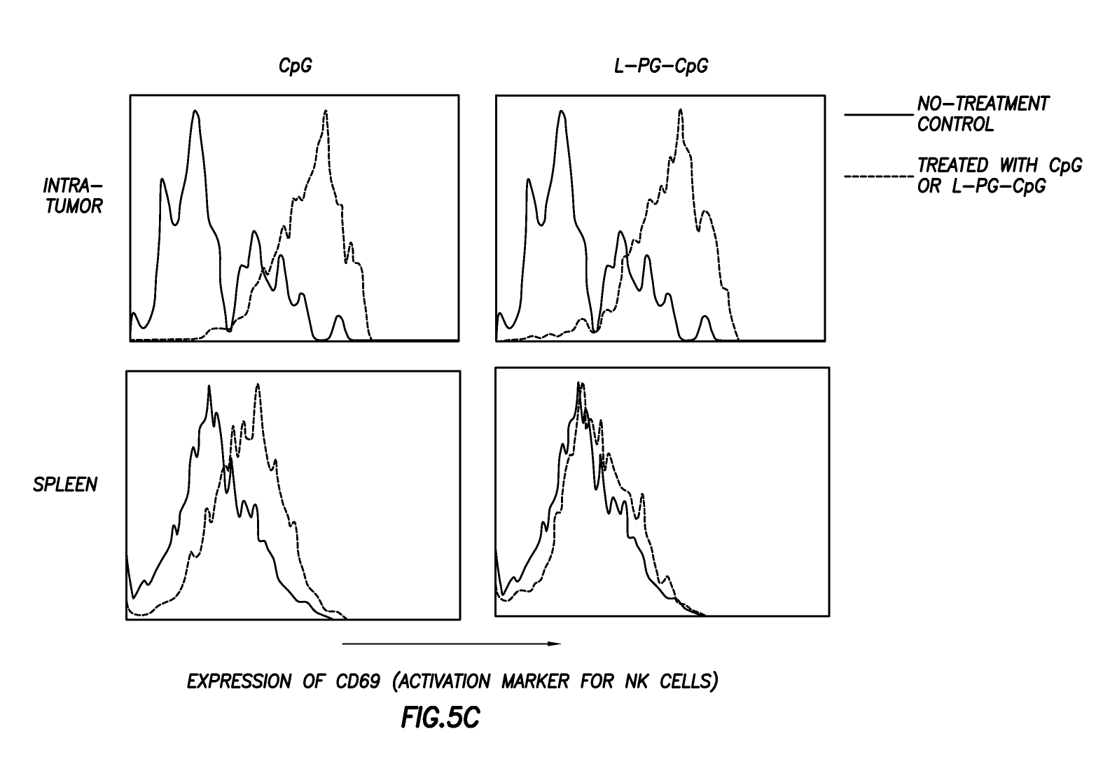

Using the subcutaneous mouse B16 melanoma model, we demonstrated that intratumoral injection of L-PG-CpG conjugate triggered significantly more antitumor activity than free CpG as a stand-alone agent against established tumors, resulting in inhibition of tumor growth (FIG. 5A). L-PG-CpG significantly enhanced the activation of tumor-nonspecific CD8 T-cell populations compared to free CpG (FIG. 5B). Significantly, while free CpG induced unspecific activation of systemic immune effector NK cells, PG-CpG did not (FIG. 5C). These results indicated that L-PG-CpG enhances the immunostimulatory potency and antitumor efficacy of CpG without causing systemic immune response.

The mechanisms that enhance the immunopotency of CpG and mediate the strong antitumor effect of L-PG-CpG are not fully understood, but several factors may contribute to this activity, including (i) a depot effect, whereby PG-CpG is retained in the tumor for a prolonged period and CpG is slowly released from the site of its injection; (ii) enhanced delivery of CpG to pDC and APC in the tumor; and (iii) co-localization of L-PG-CpG and tumor antigens within the tumor for antigen presentation. Spontaneous tumor cell death/remodeling may provide “danger” signals, which may form physical associations between L-PG and the tumor associated antigens, resulting in enhanced anticancer immunity. We have performed several preliminary studies to evaluate the potential contributions of these possible scenarios.

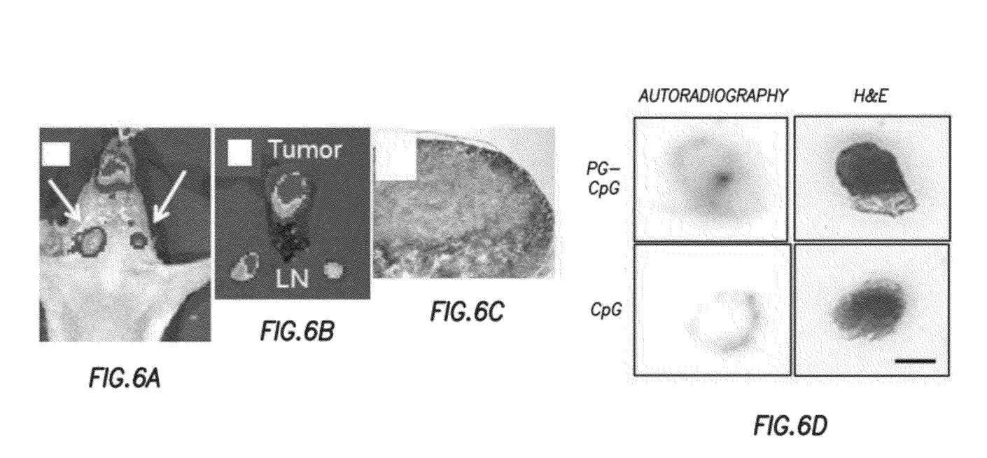

L-PG Delivery System Enhances Intratumoral Retention after Intratumoral Injection
To assess in vivo distribution and intratumoral distribution of L-PG, we used near-infrared fluorescence (NIRF) imaging with L-PG-NIR (FIG. 3) as a surrogate for L-PG-CpG and autoradiography with ¹¹¹In-labeled L-PG-CpG (FIG. 6). After intratumoral injection, L-PG-NIR was largely retained at the injection site. A significant fraction of the injected dose was also transported to the draining lymph nodes (FIG. 6A-C). In B16 melanoma, more ¹¹¹In-labeled L-PG-CpG than CpG was retained inside the tumor, and L-PG-CpG was more broadly distributed throughout the tumor, whereas CpG was localized primarily in the peritumoral area (FIG. 6D).

### Enhanced Antitumor Effect of Combined Intratumoral CpG Treatment and Reagents Targeting Costimulatory Pathways.

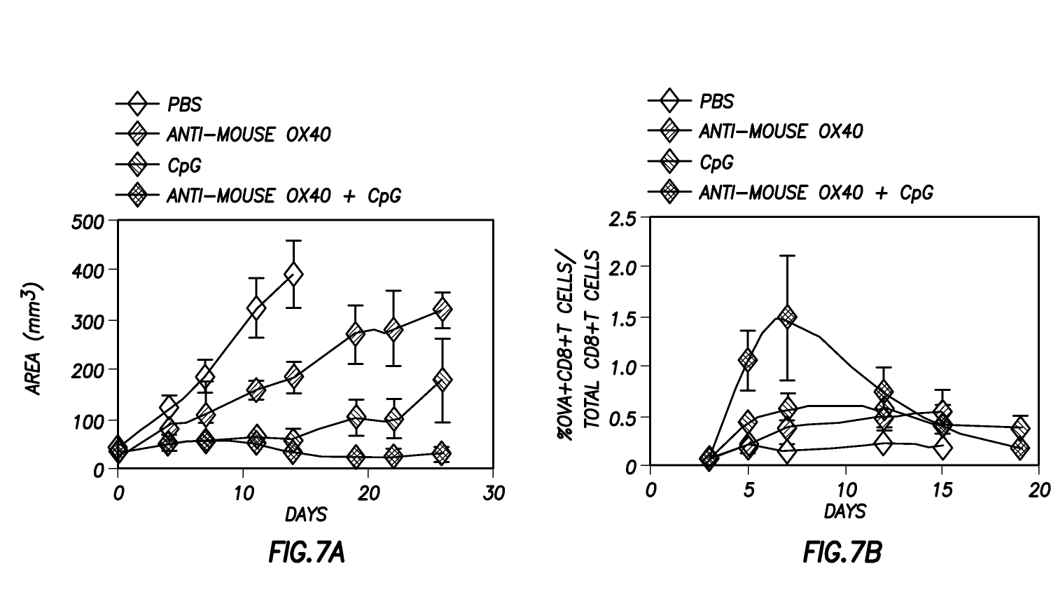

We investigated whether the combinations of intratumoral injection of CpG and systemic antibody therapy targeting costimulatory pathways leads to robust antitumor activity. Our preliminary studies demonstrated that, for example, the combination of OX40 antibody and CpG generated a significantly enhanced CTL response and antitumor effect compared to CpG alone (FIG. 7). Based on these results, we further explore novel approaches targeting both positive and negative costimulatory molecules to boost T-cell immunity to melanoma using the best nano-CpG as described below.

Significantly Less L-PG-CpG than CpG is Taken Up by the Liver and Spleen after Intravenous Administration.

In addition to identifying PG-based nano-CpG with significant immunostimulatory activity after intratumoral injection, the PG-based nano-CpG was developed for tumor-specific immune response without systemic activation. To test the feasibility, we conducted three key experiments to address the following questions: 1) whether L-PG would significantly reduction the uptake of CpG in the liver and the spleen; 2) whether L-PG polymer that is distributed to the tumor is taken up by macrophages in the tumor; and 3) whether L-PG-CpG targeted to melanoma cells can be processed by the melanoma cells to elicit robust immune response. The answers to these questions are summarized in the next three studies.

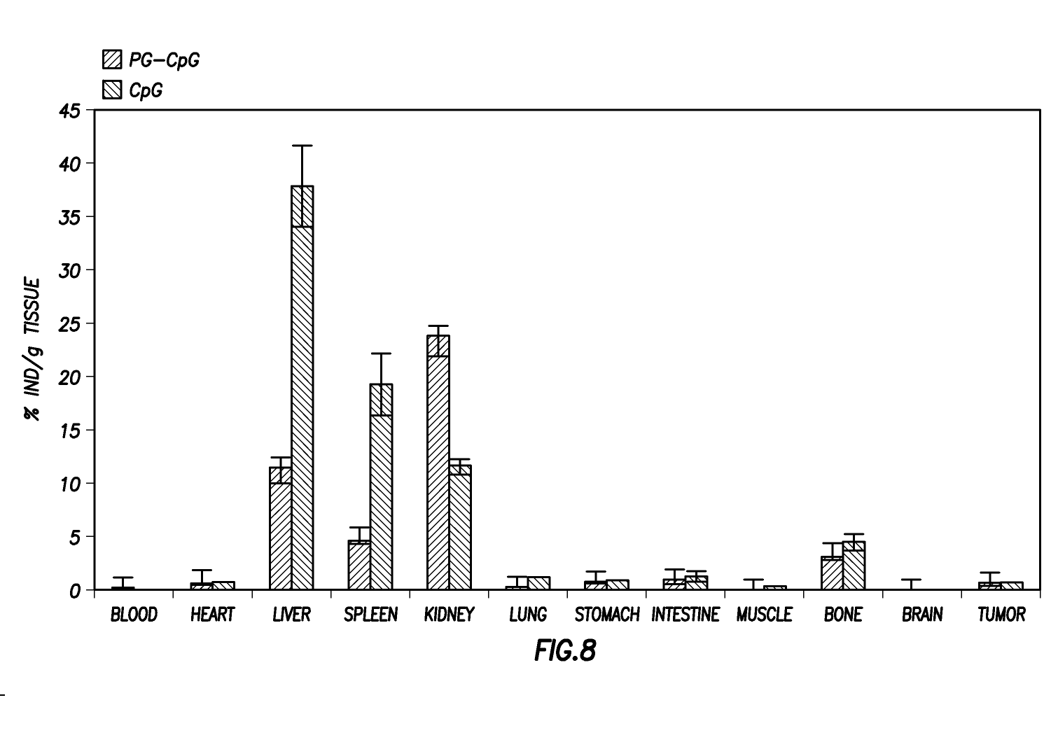

To compare bio-distribution of CpG versus PG-CpG after intravenous injection, we labeled both compounds with ¹¹¹In, a gamma emitter with a half-life of 3 days. As shown in FIG. 8, significantly less PG-CpG than CpG was taken up by the liver and the spleen. This result suggested that L-PG is a promising nanocarrier for targeted delivery of CpG after systemic administration.

L-PG Polymer is Taken Up by Tumor-Associated Macrophages after Intravenous Injection.

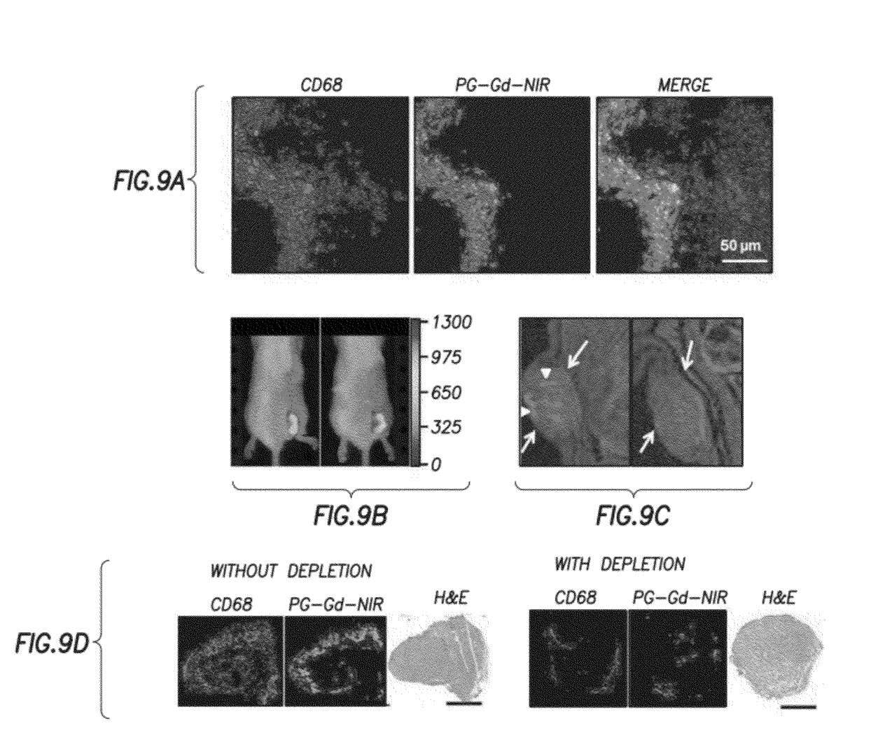

Using L-PG-Gd-NIR (FIG. 3) labeled with both Gd and NIR dye for noninvasive monitoring of the tissue and intratumoral distribution with magnetic resonance and NIRF imaging, we found that PG-Gd-NIR was taken up by tumor-associated macrophages following administration by the intravenous route (FIG. 9). PG-Gd-NIR co-localized with CD-68+ in C6 glioma in nude rats (FIG. 9A). Moreover, depletion of macrophages resulted in significant reduction in the uptake of PG-Gd-NIR in the tumors (FIG. 9B-D). Quantification showed more than 100-fold reduction in fluorescent intensity of the tumor region after depletion of macrophages with clodronate liposomes. The data indicate that PG-Gd-NIR is delivered to macrophages/APC in the tumors.

### PG-CpG is Taken Up and Processed by B16 Melanoma Cells.

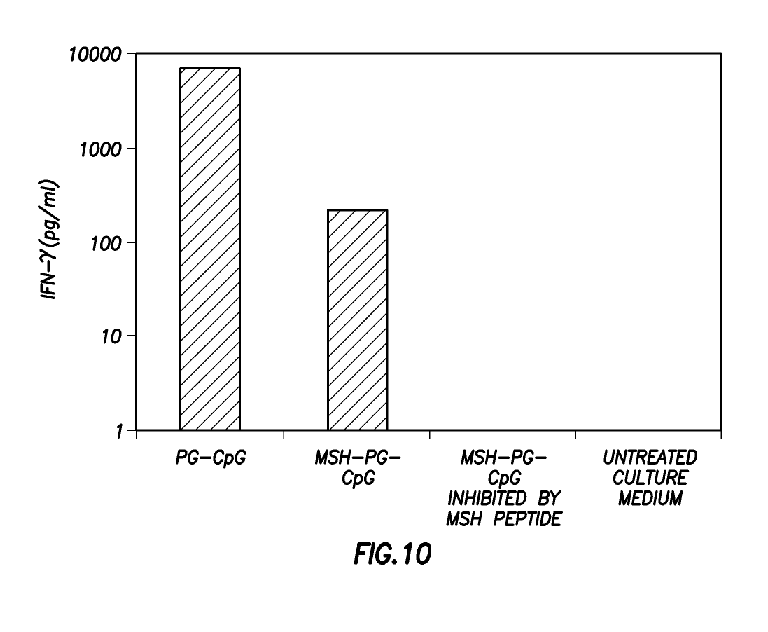

We synthesized MSH-L-PG-CpG to test whether L-PG-CpG taken up by melanoma cells via receptor-mediated endocytosis could be processed and maintain its immunostimulatory activity. As shown in FIG. 10, MSH-L-PG-CpG taken up by B16 cells was able to release active CpG into the culture media to induce production of IFN-γ by mouse splenocytes. B16 cells co-treated with an excess of MSH peptide blocked the uptake of MSH-L-PG-CpG and abolished subsequent IFN-γ production by splenocytes, which implies that melanoma cells were able of processing MSH-L-PG-CpG and present active CpG species to pDC in the tumors. These results thus validated targeting CpG to melanoma cells as a novel strategy for the induction of tumor-specific immunity. Interestingly, PG-CpG without MSH could also be efficiently taken up by B16F10 melanoma cells independently of MC1R-mediated uptake. Clearly, more studies are needed to elucidate cellular trafficking and processing of L-PG-CpG and MSH-L-PG-CpG in melanoma cells.

### Implications of Preliminary Data.

Taken together, our preliminary results indicate that L-PG is a novel and promising CpG carrier for immunostimulatory TLR agonists for the treatment of melanoma. L-PG could significantly enhance the activity of CpG by prolonging its tumor retention and thus acting as drug reservoirs allowing sustained release of CpG. APC, including macrophages and DC, avidly accumulate L-PG-based polymeric contrast agent. Targeting L-PG-CpG to melanoma cells and creating a “smart” L-PG-based CpG delivery system that only releases CpG upon the action of melanoma-specific tyrosinase is also possible, which may allow further improvement in tumor-specific CTL response. Because the immune system is complex, it is necessary to explore combination of nano-CpG with other molecules that modulate the costimulatory pathways and interrogate the tumor microenvironment. With these combined approaches, we gain insight into localized immune effects prevalent in the tumor, and can develop effective antitumor immunotherapy that can be translated to the clinic to have a significant positive impact on the care of patients with melanoma.

### Prophetic Example 1

### To Determine the Optimal Physicochemical Characteristics of PG-CpG to their Anticancer Effect Following Intratumoral Injection

Following intratumoral injection, PG-based CpG nanoconstructs with optimal physicochemical properties activate pDC locally, without inducing systemic immune response, leading to potent immunotherapeutic effect.

### Rationale and Overall Strategy.

In preliminary studies above, we have shown significantly enhanced antitumor activity of intratumorally injected L-PG-CpG compared to intratumorally injected free CpG. However, the mechanisms that enhance the immunopotency of CpG and mediate strong antitumor effect of PG-CpG are not fully understood. Several factors may contribute to this activity, including (i) a depot effect whereby L-PG-CpG is retained in the tumor for a prolonged period of time and CpG is slowly released from the site of its injection; (ii) enhanced delivery of CpG to APC and DC in the tumor; (iii) co-delivery of L-PG-CpG and TAA to DC in the tumor. Spontaneous tumor cell death/remodeling may provide TAA, which then form physical associations between LPG polymer and the antigens in the tumor, resulting in enhanced anticancer immunity.

A systematic approach is necessary in order to fully understand the possible role played by these factors that may have contributed to enhanced antitumor activity of L-PG-CpG. In addition, data obtained from these types of studies are expected to lead to the identification of physicochemical characteristics of PG polymers critical for potent immune effector cell activation after intratumoral injection of PG-based CpG nanoconstructs, which will help designing the future generation of advanced CpG delivery systems. In contrast to other studies in which CpG was incorporated inside nanoparticles, CpG will be conjugated to water-soluble L-PG, which may be advantageous in ensuring that, either in its intact form or as active species result from polymer degradation, PG-CpG is readily accessible to TLR9 binding in the endosome.

The mechanisms of action for enhanced antitumor activity of intratumorally injected PG-CpG can be investigated by systematically examining how the size (molecular weight; MW), charge, and degradability of polymers affect their retention in B16 melanoma and their uptake in pDC in the tumor in particular. The tumor retention of PG-CpG and pDC uptake of PG-CpG can be associated with enhanced innate and adaptive immunoresponses.

Table 1 below summarizes the PG-based polymers synthesized and tested for the proposed studies. Monoglutamate-CpG conjugate is included as a control. The tumor retention of L-PG-CpG after intratumoral injection is expected to be governed by its MW, because the diffusion coefficient of a molecule scales approximately as the inverse of the cube root of the MW. Molecules of smaller size may be rapidly cleared from the injection site, whereas macromolecules of larger size may be mostly confined at the injection site with very heterogeneous intratumoral distribution. In addition, the binding affinity between polyanionic PG and positively charged proteins in the tumor should also increase as a function of increasing polyanion chain length. Degradation and release of CpG from PG-CpG will be instituted by two methods: polymer backbone degradation and introduction of hydrolytically labile linker between CpG and PG polymers. Thus, CpG will be conjugated to nondegradable poly(D-glutamic acid) (D-PG), and copolymers of L-PG with L-tyrosine and L-alanine [poly(L-Glu-Tyr) and poly(L-Glu-Ala)], which degrade faster than L-PG. Chiu H-C, et al., Lysosomal Degradability of Poly(alpha-amino acids), J Biomed Mat Res 34:381-92, 1997. CpG will also be conjugated to L-PG and D-PG through acid-labile linkers that will rapidly release CpG in the acidic environment of endosomes.

Finally, conjugated CpG to degradable but neutrally charged poly(hydroxypropyl L-glutamate)(L-PHPG) allows for examination of the possible role of physical interaction between polyanionic L-PG-CpG and positively charged proteins from the tumor itself. Id. Hence, the impact of CpG delivery on both the innate and adaptive immune responses in vivo can be examined.

However, given that in vitro studies have limited capacity to predict the efficacy of vaccines due to the complexity of the immune system, our focus is on in vivo evaluation using the syngeneic B16 melanoma model. B16 melanoma in C57BL/6 mice has been used in many preclinical melanoma studies. Poor immunogenicity and the fact that B16 cells express very low amounts of MHC class 1 molecules make the B16 model a challenging model for T-cell-based immunotherapy. Mansour M, et al., Therapy of Established B16-F10 Melanoma Tumors by a Single Vaccination of CTL/T helper Peptides in VacciMax(R), 2007. p. 20. Various approaches have been adopted for the generation of CTL responses against B16, including co-delivery of isolated TAA and CpG in nanoparticles. However, our proposed studies are significantly different from these other studies in that no purified TAA will be mixed with PG-CpG prior to injection. A more realistic and clinically relevant melanoma antigens in situ from the tumor itself should be used.

TABLE 1
PG-based polymers to be synthesized and tested.

| | | | Implications on |
|---|---|---|---|
| Polymers Structures | | Properties Compared | CpG Function |
| Glutamate-CpG | | Monomer | Serve as monomer |
| | | | control |
| L-PG- | MW = 2K | Molecular weight/ | Retain CpG in |
| CpG | MW = 20K | degradability | the tumor |
| | MW = 50K | | |
| | MW = 100K | | |

| | | |
|---|---|---|
| D-PG-CpG | Non-degradable | Examine the effect |
| | | of degradation rate |
| Poly(L-Glu-Tyr) | Degrade faster | Degradable/negative |
| | than L-PG | charge |
| Poly(L-Glu-Ala) | Degrade faster | Examine the effect |
| | than L-PG | of degradation rate |
| L-PHPG | Neutral polymer | Examine the effect |
| | | of charge |
| L-PG-ketal-CpG | pH sensitive release | Examine the effect |
| | of CpG & backbone | of release rate |
| | degradation | |
| D-PG-ketal-CpG | pH sensitive release | Examine the effect |
| | of CpG & no backbone | of release rate |
| | degradation | |

### Synthesis

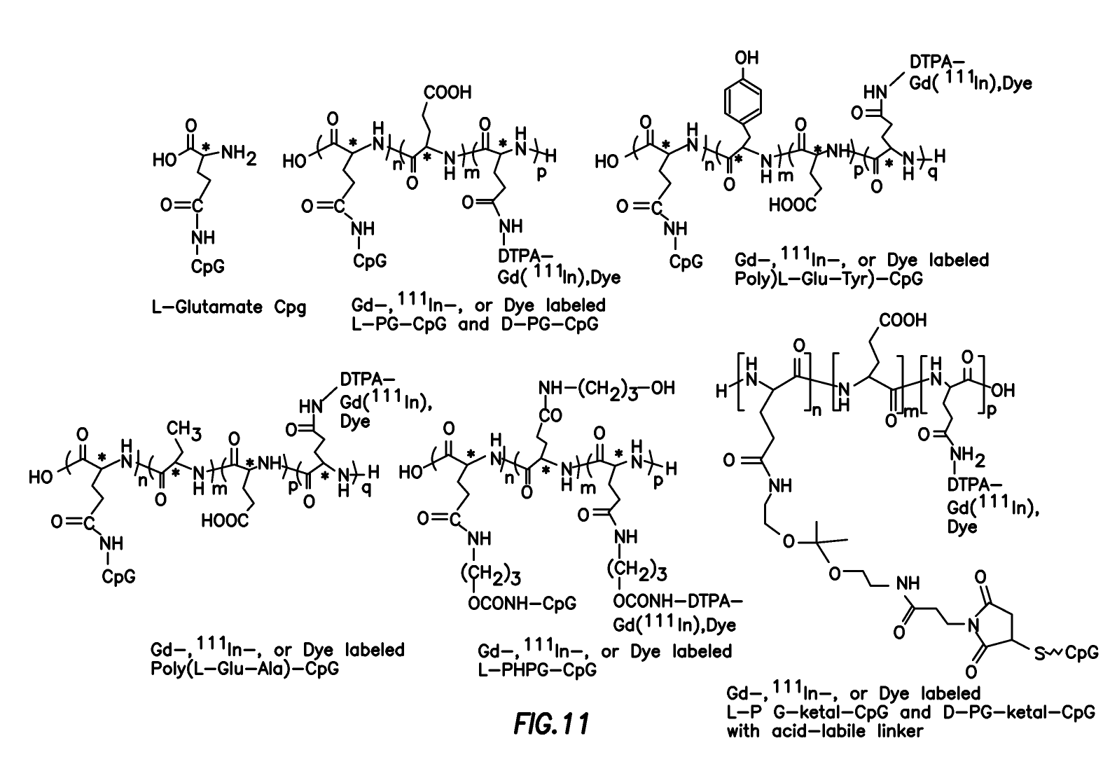

In FIG. 11, the structures of the target compounds are summarized. The monomeric L-glutamate CpG will be synthesized using 1,3-dlisopropylcarbodiimide-mediated coupling reaction between the side chain carboxyl group of Boc-Glu(OH)—OtBu and the amino group of 3′-NH₂-CpG in the presence of N-hydroxybenzotriazole. The Boc and tert-butyl protecting groups of the resulting product will be removed with trifluoroacetic acid.

L-PG is usually obtained from poly(γ-benzyl-L-glutamate) (L-PBLG) by removing the benzyl protecting group with hydrogen bromide. For the preparation of homo-polymers and random copolymers, triethylamine-initiated polymerization of the N-carboxylanhydrides (NCA) of γ-benzyl-L-glutamate (NCA-L-Glu) and the corresponding amino acids (i.e. NCA-D-Glu, NCA-L-Tyr, or NCA-L-Ana) are the most frequently used methods. Li C., Poly(L-glutamic acid)—Anticancer Drug Conjugates, Adv Drug Deliv Rev 54:695-713, 2002. This method typically yields polyamino acids of broad MW distributions (polydispersity>2.0). A few controlled NCA polymerizations have been reported over the last decade, including the use of transition metal complex and hexamethyldisilazane as initiators. Deming T J., Cobalt and Iron Initiators for the Controlled Polymerization of Alpha-Amino Acid-N-Carboxyanhydrides, Macromolecules 32:4500-2, 1999; Lu H, Cheng J., Hexamethyldisilazane-Mediated Controlled Polymerization of Alpha-Amino Acid N-Carboxyanhydrides, J Am Chem Soc 129:14114-5, 2007. The reaction using hexamethyldisilazane as an initiator is particularly attractive because the method readily affords polyamino acids with predictable MWs and narrow MW distributions.

Therefore, this method is used to synthesize poly(L-Glu-Tyr) and poly(L-Glu-Ala) with Glu:Tyr and Glu:Ala molar ratio of 5:1. Studies by Chiu et al. have shown that copolymers of with L-Tyr and L-Ala degrade about 3-fold faster than homopolymer L-PG. Chiu H-C, et al., Lysosomal Degradability of Poly(alpha-amino acids), J Biomed Mat Res 34:381-92, 1997. To facilitate in vitro and in vivo imaging studies, DTPA or a suitable fluorescent dye will be conjugated to L-PG of different MWs (MW=2K, 20K, 50K, or 100K), D-PG (MW=50K), poly(L-Glu-Tyr) (MW=50K), and poly(L-Glu-Ala) (MW=50K) according to our previously reported procedures. 3′-amino-CpG (ODC 2216) will be conjugated to these conjugates at the final stage in 2-morpholinoethanesulfonic acid buffer using the water-soluble coupling agent 1-ethyl-3-(3-dimethylaminopropyl)carbodiimide). Melancon M P, et al., A Novel Method for Imaging In Vivo Degradation of Poly(L-Glutamic Acid), A Biodegradable Drug Carrier, Pharm Res 24:1217-24, 2007; Melancon M P, et al., Development of a Macromolecular Dual-Modality MR-Optical Imaging for Sentinel Lymph Node Mapping, Invest Radiol 42:569-78, 2007; Wen X, et al., Synthesis and Characterization of Poly(L-glutamic acid) Gadolinium Chelate: A New Biodegradable MRI Contrast Agent, Bioconjug Chem 15:1408-15, 2004. The resulting conjugates will be labeled with Gd for MRI and ¹¹¹In for nuclear imaging studies. The choice of dye will depend on the intention of the study. For in viva imaging, the NIR dye that emits fluorescent signal at 813 nm will be used. For flow cytometry studies, a FACS-compatible dye (i.e. AlexaFluor488) will be chosen. The conjugates will be purified on an ÄKTA fast protein liquid chromatography system equipped with a superdex-75 SEC column and eluted with PBS buffer.

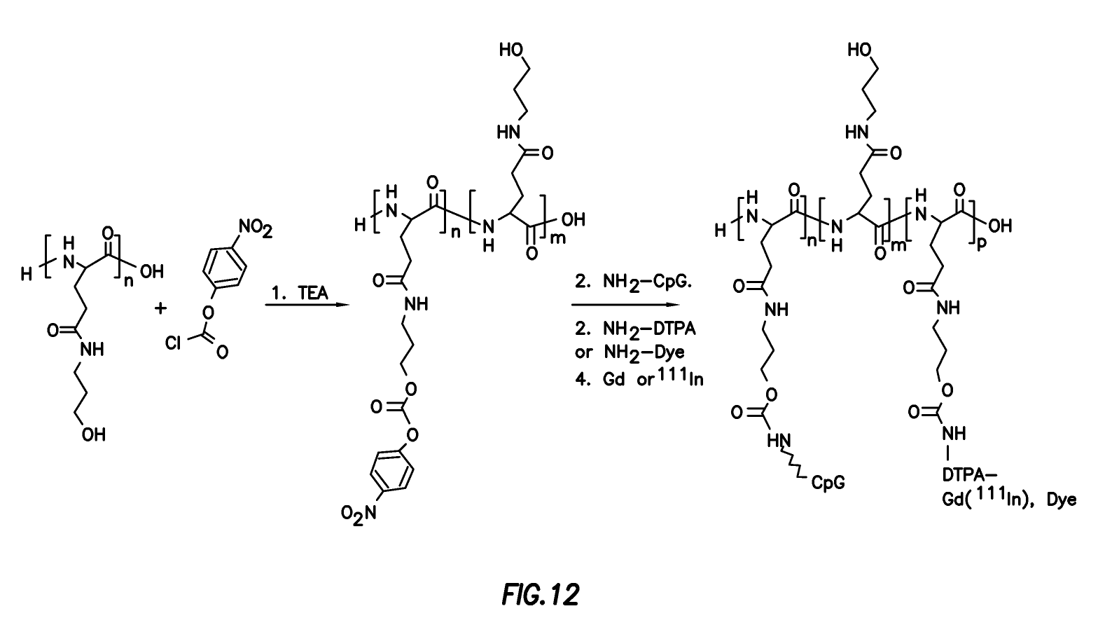

For the synthesis of L-PHPG-CpG, L-PHPG will be Obtained by aminolysis of L-PBLG with 3-aminopropanol. After activation of the hydroxyl groups in L-PHPG with p-nitrophenyl chloroformate, the polymer will be treated with amine-terminated DTPA or dye molecules via a carbamate linkage. CpG will then be conjugated to the polymer, followed by chelation to Gd or ¹¹¹In to afford Gd-, ¹¹¹In-, or dye-labeled L-PHPG-CpG (FIG. 12).

The release of CpG from PG-CpG nanoconstructs with CpG linked to PG via stable amide or carbamate bonds relies on degradation of the PG backbone to release monomeric and oligomeric glutamate CpG by cathepsin B and/or other enzymes in the tumor. Because enzymatic activity may vary from tumor to tumor and from patient to patient, we wish to design a system whereby the release of CpG is not dependent on the enzymatic activity, but instead depends on the acidic environment of the endolysosomal compartments. This approach may reduce variation in CpG delivery and increase intracellular delivery of CpG. To achieve this aim, we will graft CpG to L-PG and D-PG through acid-labile ketal linkers (FIG. 11). Endosomes and lysosomes exist at acidic pH values between 5.0 and 6.5, in contrast to the cytoplasm, which is at pH 7.4. Acid-labile linkage has been employed in designing pH-responsive delivery of oligonucleotides and protein vaccines. Knorr V, Ogris M, Wagner E., An Acid Sensitive Ketal-based Polyethylene Glycol-Oligoethylenimine Copolymer Mediates Improved Transfection Efficiency at Reduced Toxicity, Pharm Res 25:2937-45, 2008; Murthy N, et al., Design and Synthesis of pH-responsive Polymeric Carriers that Target Uptake and Enhance the Intracellular Delivery of Oligonucleotides, J Control Release 89:365-74, 2003; Murthy N, et al., A Macromolecular Delivery Vehicle for Protein-Based Vaccines: Acid-Degradable Protein-Loaded Microgels, Proc Natl Acad Sci USA 100:4995-5000, 2003.

We will adopt similar ketal linker chemistry in our design to conjugate CpG to both L-PG and D-PG. D-PG is included here so that a possible effect of backbone degradation on CpG release can be excluded. Starting from acetone-bis-(aminoethyl)ketal, we will introduce N-maleimide to one end of the diamine ketal to give acetone-(maleimidoaminoethyl)ketal. This linker will then be conjugated to PG using carbodiimide-mediated coupling reaction, followed by attachment of 3′-SH-CpG to the maleimido-linker. The imaging probes will be conjugated to the polymers as described before.

### Characterization

The resulting polymeric conjugates will be characterized with regard to 1) structure and composition ratios of copolymers; 2) the number of CpG, DTPA-Gd, and dyes attached to each polymer; 3) the MWs and MW distributions; and 4) degradability. The composition ratios of the copolymers will be characterized by ¹H-nuclear magnetic resonance. The number of CpG, DTPA-Gd, and dyes attached to each polymer will be determined by subtracting the amount of the recovered molecules in the reaction mixture from the amount of the starting materials. If necessary, the number of CpG, DTPA-Gd, and dye molecules may also be determined from amino acid analysis after complete hydrolysis of the polymers. Gd contents will be determined by elemental analysis. The MW and MW distribution of each polymeric conjugate will be measured by gel permeation chromatography (GPC) using a system equipped with a Viscotek E-Z^{pro} triple detector (Viscotek, Houston, Tex.) that records refractive index, viscosity, and light-scattering signals. The enzymatic degradation of each polymeric conjugate will be performed in the presence or absence of cathepsin B using GPC according to Wen et al. Wen X, et al., Synthesis and Characterization of Poly(L-glutamic acid) Gadolinium Chelate: A New Biodegradable MRI Contrast Agent, Bioconjug Chem 15:1408-15, 2004. The decrease in peak area of each polymeric conjugate will be monitored with time and expressed as “percentage of degradation,” The hydrolytic degradation of PG-ketal-CpG conjugates will be studied by analyzing the release of CpG over time at pH 5, pH 6, and pH 7.4 using a high-performance liquid chromatography-mass spectrometry system.

For evaluation of cellular uptake and trafficking of newly synthesized CpG-bound nanoconstructs in vitro and their retention in vivo after intratumoral injection into B16 tumor, macrophages will be generated from bone marrow of C57BL/6 mice according to our published protocol. Thapa P, Zhang G, Xia C, et al., Nanoparticle Formulated Alpha-Galactosylceramide Activates NKT Cells Without Inducing Anergy, Vaccine 27:3484-8, 2009. To study the internalization of polymer-CpG, macrophages will be pulsed with fluorescent labeled polymers for 1 hour, fixed, and stained by monoclonal antibodies toward EEA (early endosome marker), Mannose-6 phosphate receptor (late endosome receptor), and LAMP1 (lysosome marker). All antibodies will be from Abcam (Cambridge, Mass.). The colocalization of polymer and different endolysosome markers will be studied by confocal microscopy.

### In Vivo Retention, Intratumoral Distribution, and Degradation:

We will use nuclear and MR imaging techniques to noninvasively assess the retention and distribution of PG-CpG nanoconstructs in B16 melanoma (total 11 preparations) (Table 1 and FIG. 11). Free CpG will be labeled with ¹¹¹In only as a control. All PG-CpG nanoconstructs will be dually labeled with Gd/¹¹¹In. Each labeled agent will be delivered by intratumoral injection into B16 melanoma grown subcutaneously in C57BL/6 mice (6-8 mm in diameter) in a single injection (100 μCi, 100 μl). The mice will be imaged with a γ-camera at various times after injection. The radioactivity in the tumor and the rest of the body will be quantified by placing a region of interest around the whole body, the liver/spleen area, and the tumor. This will allow us to measure the amount of CpG and PG-CpG cleared from the tumor over time in the same mice. Because MRI provides excellent spatial resolution, we will also use MRI to monitor the intratumoral distribution of PG-CpG nanoconstructs at different times. By the end of the imaging sessions (3 days after injection), mice will be killed. Liver, spleen, draining lymph nodes, and tumor will be removed and counted for radioactivity. Tumor retention will be expressed as a percentage of the injected dose. Autoradiographic studies will be performed on all excised tumors. The uniformity of intratumoral distribution will be analyzed by measuring the ratio of the radioactive area to the whole tumor area expressed as a percentage. CpG and PG-based CpG nanoconstructs will be ranked according to their tumor retention (%) as well as extent of intratumoral distribution (%). To examine the biodegradation of polymers in B16 melanoma, halt of each exercised tumor tissue will be processed for GPC analysis using a NaCl crystal detector to monitor the elution of radioactive intact polymers and polymer fragments from the column. (12 groups×10 mice).

Evaluation of the Innate and Acquired Immunity Induced by CpG and PG-Based CpG Nanoconstructs after Intratumoral Injection.

### Cellular Uptake In Vivo

To assess the uptake of CpG-bound PG nanoconstructs in different immune cell populations, L-Glu-dye monomer and each PG-CpG-dye conjugate (dye=AlexaFluor 647) shown in FIG. 11 will be injected intratumorally into B16 tumors (n=10). CpG-dye will be used as a control. At 1, 3, and 7 days later, CpG uptake in CD11c+ DC and CD11b+F480+ macrophages will be measured in tumor, draining lymph nodes, and spleen by flow cytometry. Because the fluorescent dyes attached to PG polymers will dissociate from CpG attached to the same polymer chains when polymer disintegrates, they directly report on the cellular uptake of polymers or polymer fragments. For this reason, we will also attach fluorescence-labeled CpG (e.g., 3′-NH₂-CpG-AlexaFluor 647, Alpha DNA, Montreal, Canada) to PG polymers shown in FIG. 11 for FACS studies of the cellular uptake of CpG and CpG in PG-CpG conjugates. The following antibodies will be use for cell labeling: CD11c for DC and CD11b and F480 for macrophages.

In addition, transgenic mice that express GFP in monocyte lineage cells (under control of the murine c-fms promoter) will be used as tumor transplant recipients, which will allow us to study the co-localization of NIR fluorescent dye-labeled polymer and monocyte-macrophages using noninvasive imaging in live animals. The animals are available from the Jackson Lab. (Bar Harbor, Me.).

### Local Cytokine Production

IFN-α, IL-12, and IFN-γ will be measured using standard ELISA kits from R&D systems (Minneapolis, Minn.).

### Delivery to APA in B16 Melanoma Activation of pDC

The delivery of polymer to tumor APC will be studied by multiple color flow cytometry using antibodies against DC (CD11c+) and macrophages (CD11b+F480+). The activation of pDC will be monitored by IHC staining of pDC (BDCA2+) with CD69.

### Melanoma-Specific CD8+ T-Cell Response in the Tumor

Tumor-specific CD8 responses will be monitored as OVA-specific tetramers (Pharmingen, San Jose, Calif.).

### Systematic Response

Systemic responses will be studied by measuring serum levels of cytokines, including IFN-α, IFN-γ, TNF-α, and IL-12. All cytokines will be measured using ELISA kits or Luminex from R&D systems.

Finally, in all of the above studies, L-PG (MW=50K) without CpG will be used as a control.

### Validation of Antitumor Activity

All CpG-bound PG together with monoglutamate CpG (total of 11 compounds) will be studies for their antitumor activity after intratumoral injection. This is because there is no clear association between the in vitro assay (stimulating lymphocytes) and in vivo antitumor activity. Saline and free CpG will serve as controls. C57BL/6 mice bearing subcutaneous B16 melanoma tumors on both legs (average diameter 4-6 mm) will be randomly assigned to 13 treatment groups (10 mice per group). Mice in each group will receive intratumoral injection with saline, CpG, and each agent listed in Table 1 on days 1, 7, and 10 at a dose of 50 μg equivalent CpG/injection (100 μl). Only 1 of the 2 tumors in each mouse will be treated. Tumor size will be measured starting at day 7 and then every 2-3 days until day 21. The longest length (a) and the length perpendicular to the longest length (b) will be used in the formula V=½a(b)² to obtain the tumor volume in mm³. On day 21, all the animals will be sacrificed, and draining lymph nodes, spleen, and both treated and untreated tumors will be removed for IHC and FACS analysis of pDC population and cell death (TUNEL). Weights of individual tumors will be recorded and used as a measure of tumor control on day 21. The untreated tumors will be used as a tool to evaluate whether the melanoma-specific CTL response is capable of displaying antitumor activity against tumors at distant sites.

### Data Analysis and Statistics

Generalized linear models will be used to analyze the intratumoral retention of nano-CpG and their uptake in pDC. Although the final form of these models cannot be determined prior to fitting the data, we note that the proposed design will have >85% power in detecting 20% difference in each pair-wise comparison. For antitumor effect study, the tumor size, measured every 3 days after tumor inoculation, will be used as the primary end point. We will initially use 5-6 mice per group to conduct the statistical analysis and determine the variance and statistic power. Additional animals (up to a total of 10 mice per group) will be added to achieve statistic significance. If we conservatively estimate the coefficient of variation in each group to be approximately 0.25, and assume that there is a two-fold reduction in tumor size, then with 10 mice per group we will have >85% power to detect a difference between groups in t-tests at the 0.005 level (assuming equal variances in each group). Comparison among groups will be performed using one-way ANOVA using SAS software version 8.0 for Microsoft Windows (SAS Institute). The significance level will be set at 0.05.

### Anticipated Results, Pitfalls and Solutions

We expect to identify physicochemical characteristics of PG polymers that are important for the potent antitumor activity of CpG-bound PG nanoconstructs. Specifically, negatively charged PG with a relatively high MW may induce a stronger immunostimulatory response because these polymers may be retained in the tumor longer and release CpG in a sustained fashion. Polymers of larger sizes may also be more readily phagocytosed by APCs than their counterparts with smaller sizes, and have a stronger interaction with positively charged proteins in melanoma. Introduction of Tyr and Ala to L-PG not only influence polymer's degradability, but may also affect their uptake by APCs and their interaction with basic proteins as well owning to increased hydrophobicity of the copolymers. An unlikely, but potential pitfall is that PG polymer may directly interact with their endosomal receptor (TLR9), without necessity CpG being released from polymer. In that case, we will identify a polymer-CpG with strongest anticancer efficacy, for further studies. The original L-PG₁-CpG and the CpG-bound PG nanoconstruct that demonstrates the best efficacy in in vivo antitumor efficacy studies (designated as PG₂-CpG) will be advanced to Aim 2 for the design and development of CpG-bound PG nanoconstructs targeted to melanoma receptors and melanoma-specific enzymes.

### Prophetic Example II

### Develop and Validate PG-CpG Nanoconstructs Actively Targeted to Melanoma Cells Through Both Receptor-Mediated Uptake and Tyrosinase-Mediated CpG Release

PG-CpG nanoconstructs that actively target melanoma cells and release CpG only upon the action of melanoma-specific tyrosinase further enhance the immunostimulatory and antitumor activities of CpG without inducing nonspecific activation of the immune system.

### Rationale and Overall Strategy

For the treatment of deadly metastatic melanoma, it is highly desirable that CpG reach every lesion throughout the body but without triggering a systemic immune response and consequent depletion of the effector T cells needed at the tumor sites. A recent study by Sharma et al. using antibody-CpG conjugate targeted to Her2/neu-positive tumor cells and our own preliminary data with L-PG-CpG targeted to melanoma cells suggest that indirectly targeting CpG to melanoma cells is a viable strategy for immunotherapy for melanoma. We propose to use two approaches to target CpG to melanoma cells.

In the first approach, we will direct CpG-bound PG nanoconstructs to melanoma cells through melanocortin type-I receptor (MC1R)-mediated uptake using α-melanocyte stimulating hormone as the homing ligand. Targeting nanoparticles to tumor-associated receptors, although attractive, cannot completely avoid uptake of nanoparticles by the liver and the spleen. In an ideal case, the CpG-bound nanoparticles, or more specifically CpG-bound PG, would release CpG solely at the tumor site upon the action of tumor-specific enzyme. In this way, the CpG-induced activation of immune effector cells at sites other than the tumor would be greatly reduced even though some of the injection nanoparticles are distributed to the secondary lymphoid organs.

Therefore, in the second approach, we will create “smart” nanoconstructs that release CpG only upon the enzymatic activation of the highly melanoma-specific enzyme tyrosinase. Once the proposed nanostructures are obtained, we will study the efficiency of selective in vivo delivery of CpG to B16 tumors after intratumoral and intravenous injections. We will then assess the immunostimulatory activities of targeted PG-PG nanoconstructs on the melanoma and the systemic immune system, again with both intratumoral and intravenous routes of administration. Successful demonstration of systemic antitumor activity without nonspecific activation of immune response may revolutionize the field of immunotherapy for melanoma as well as other metastatic solid tumors.

### Research Design

### Synthesis and Characterization of CpG Targeted to MC1R

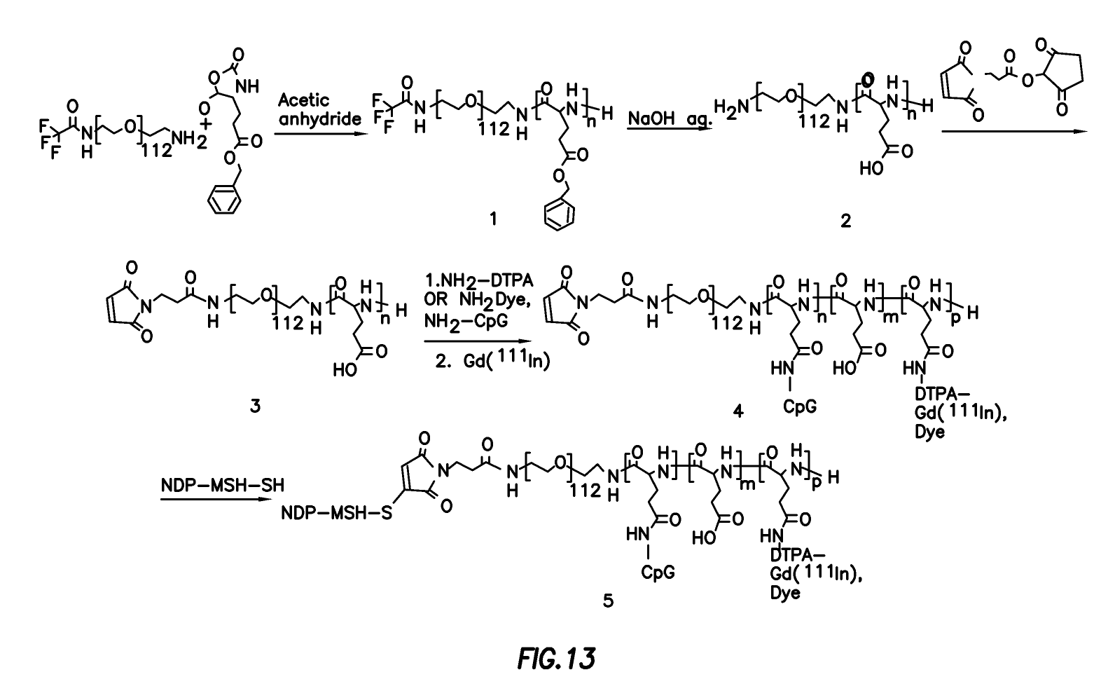

We have successfully demonstrated targeted delivery of hollow gold nanospheres to MC1R in B16 melanoma using α-MSH analogue NDP-MSH (Cys-Ser-Tyr-Ser-Nle-Glu-His-d-Phe-Arg-Trp-Gly-Lys-Pro-Val-NH₂) as the homing ligand, in which NDP-MSH was attached to the surface of gold nanospheres through polyethylene glycol (PEG) linkers. Here, we will use a similar strategy, attaching NDP-MSH to the L-PG chain through a PEG linker to increase the accessibility of the NDP-MSH peptide to MC1R as shown in FIG. 13.

Briefly, block copolymer PEG-PBLG (polymer 1) will be prepared through ring-opening polymerization of L-Glu(OBzl)-NCA using trifluoroacetamide-PEG-amine as the initiator. Subsequent deprotection in NaOH aq. solution and activation of the terminus amine with N-succinimidyl-3-maleimidopropionate will give polymer 3. NH₂-DTPA or NH₂-dye and NH₂-CpG will then be conjugated to polymer 3 using the same procedures described in section 6.1.3.a, followed by introduction of SH-NDP-MSH to yield the proposed polymer (FIG. 13). The resulting polymer 5 will be characterized as described in section 6.1.3a. Similar methods will be used for the synthesis of MC1R-targeted nanoconstructs from PG₂-CpG identified above in Prophetic Example I.

### Cell Uptake and Trafficking

Cell uptake of polymer 5 and the corresponding Gd(¹¹¹In) or dye-labeled NDP-MSH-PEG-PG₂-CpG in B16/F10 cells will be studied according to our reported method. Lu W, Xiong C, Zhang G, et al. Targeted Photothermal Ablation of Murine Melanomas With Melanocyte-Stimulating Hormone Analog-Conjugated Hollow Gold Nanospheres, Clin Cancer Res 15:876-86, 2009. Briefly, cells will be incubated with each AlexFluor647-tagged test compound at 37° C. for 1 h, followed by fixation in 4% paraformaldehyde. For inhibition study, the cells will be incubated with free NDP-MSH (200 μg/mL) for 30 min before addition of each compound. After washing and fixation, cell nuclei will be stained with DAPI. To evaluate β-arrestin activation and recruitment, cells will be treated with AlexFluor647-tagged 5 or 6 for 15 min and then subjected to β-arrestin immunohistostaining with goat anti-β-arrestin-2 polyclonal antibodies and donkey anti-goat IgG tetramethyl rhodamine isothiocyanate conjugate. The cellular fluorescence will be examined under a Zeiss Axio Observer.Z1 fluorescence microscope. AlexFluor647-tagged L-PG-CpG or PG2-CpG without the horning ligand will be used as controls.

### Synthesis and Characterization of Tyrosinase-Activable CpG-bound Nanoconstructs

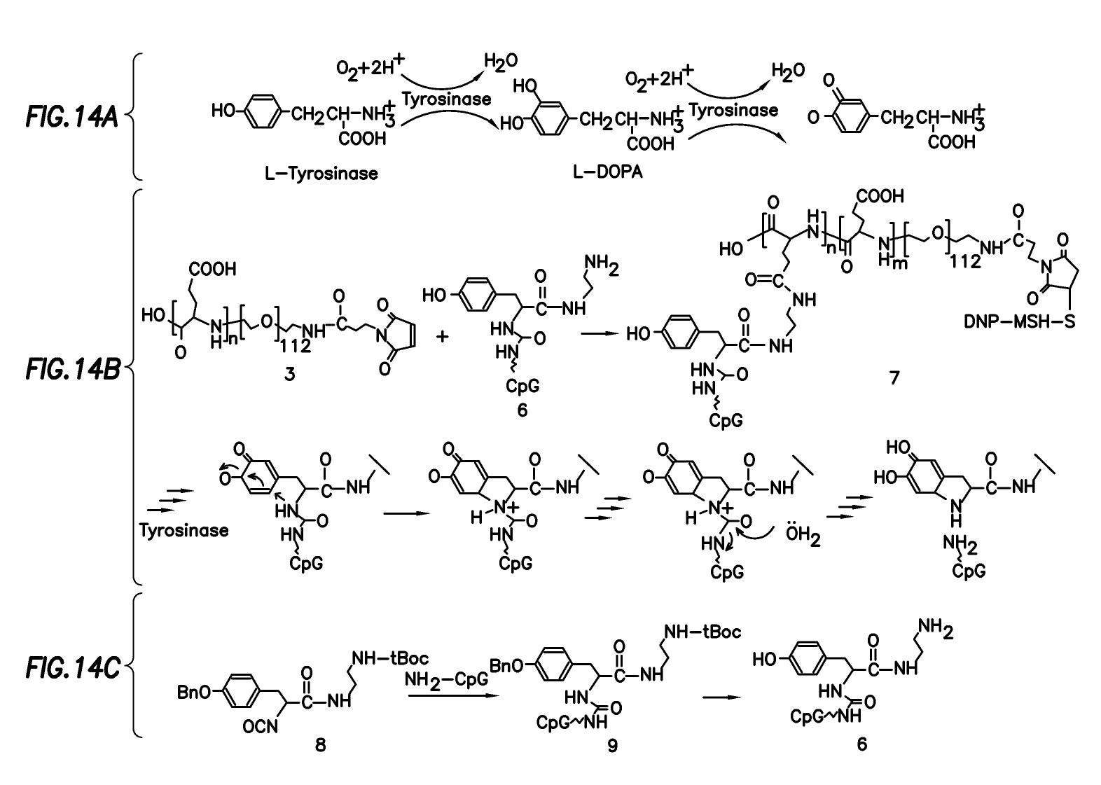

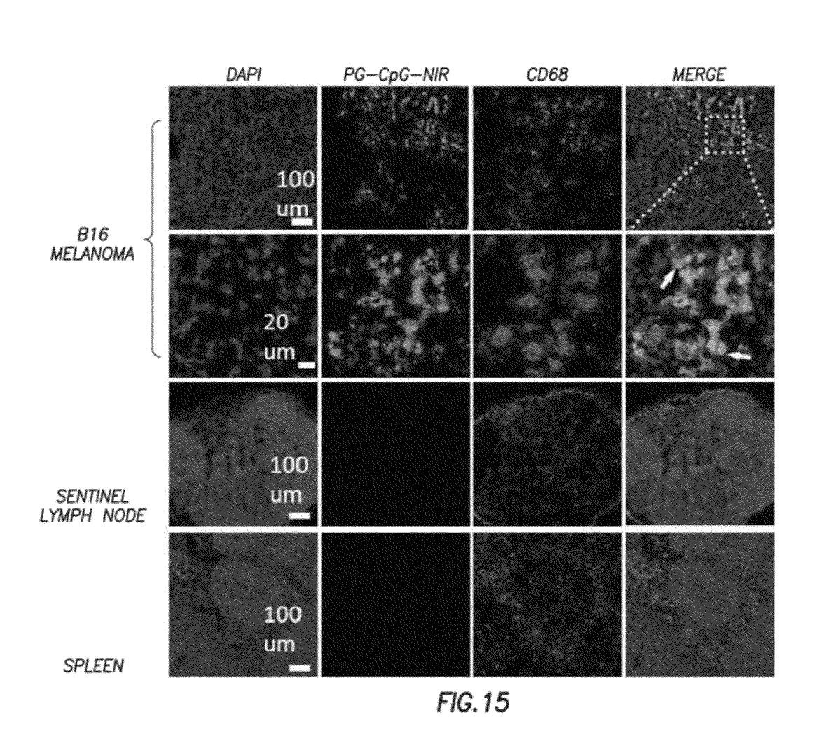

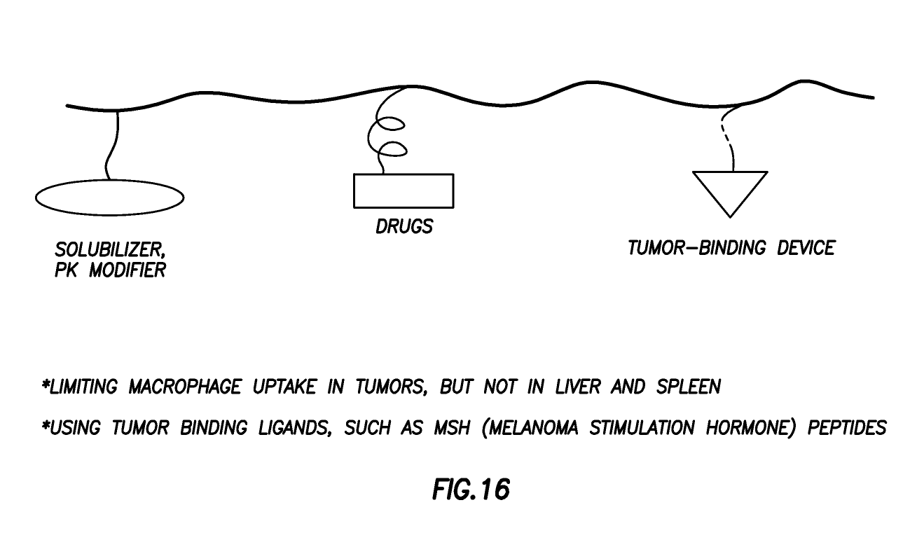

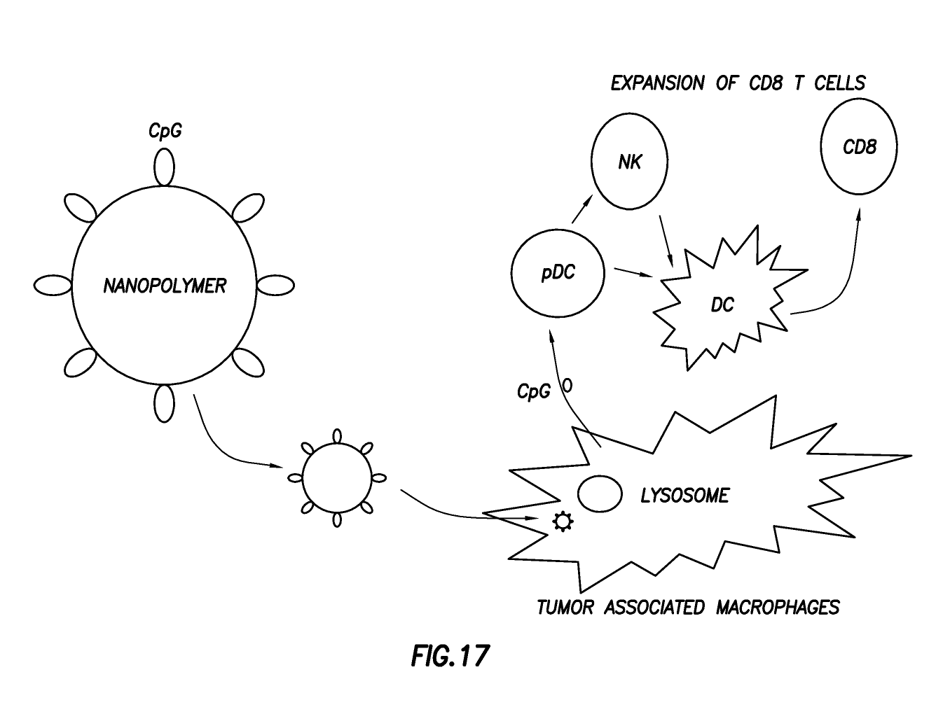

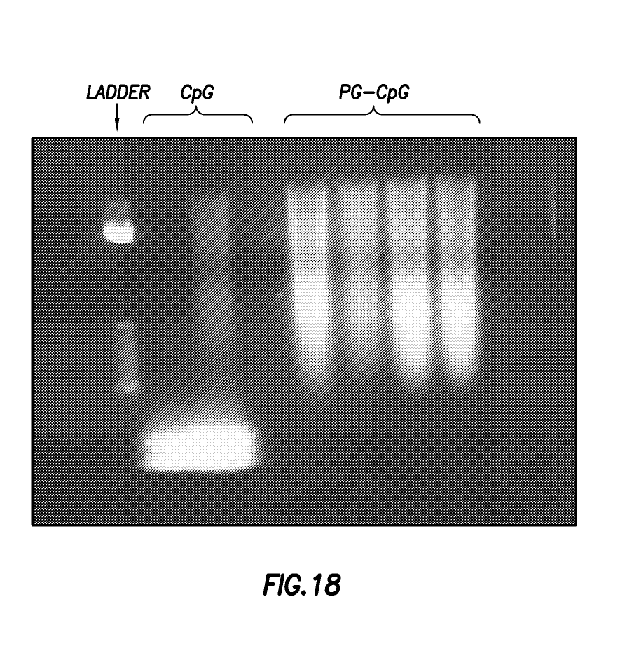

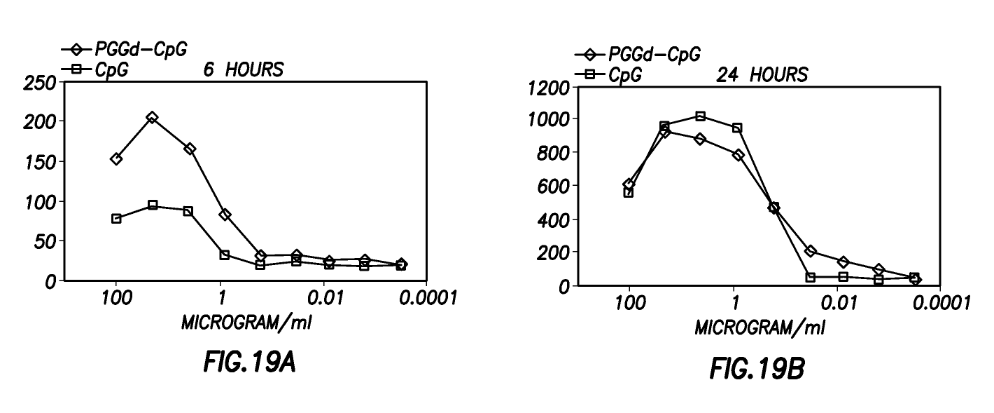

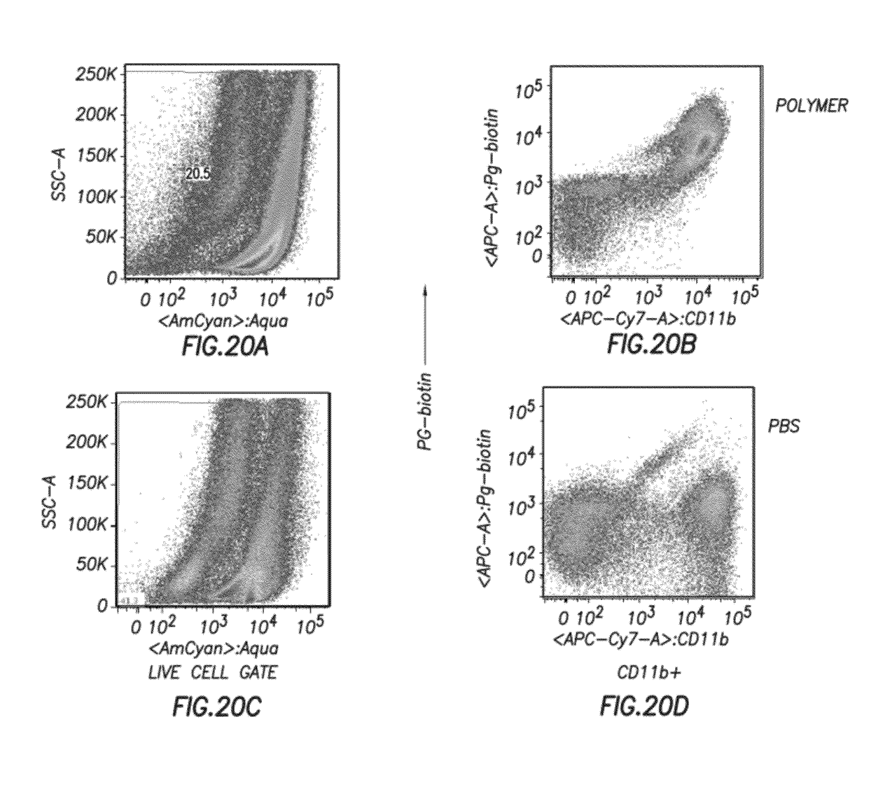

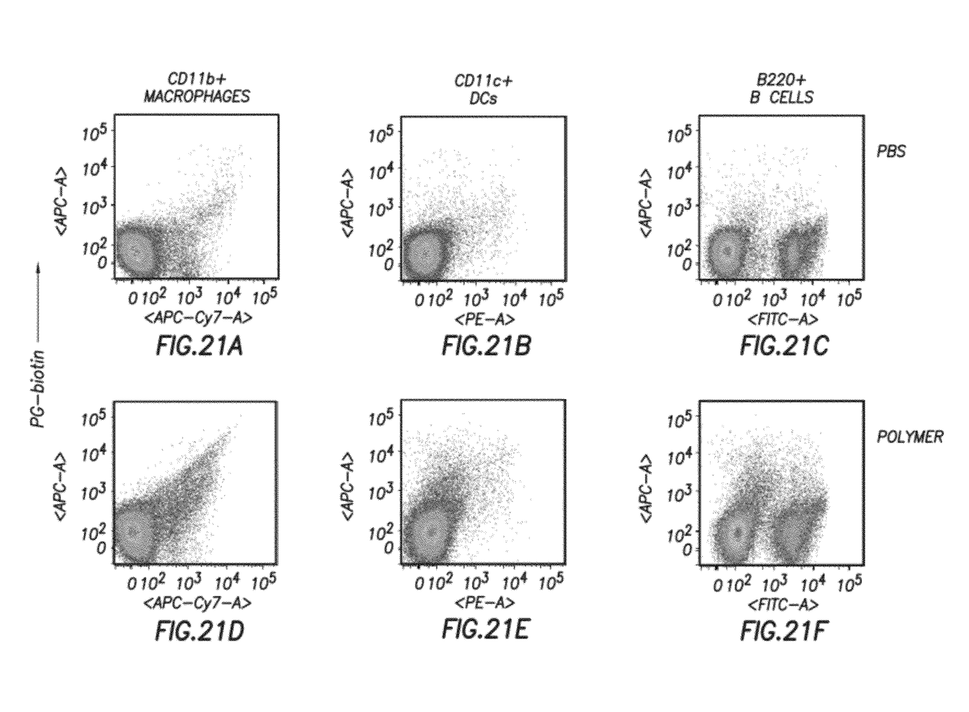

Tyrosine is the natural substrate of tyrosinase, with oxidation occurring to afford the corresponding L-DOPA and orthoquinone (FIG. 14A). Previous studies have shown that tyrosinase can be used to mediate the release of cytotoxic agents from carbamate and urea prodrugs via a cyclization-drug release mechanism. It is envisaged that the polymeric CpG prodrug 7 targeted to MC1R can be formed from the attachment of the Tyr-CpG intermediate 6 to maleimide terminated PEG-PG copolymer 3 through a urea linker, followed by Michael-addition reaction to introduce SH-DNP-MSH (FIG. 14B). Upon tyrosinase-mediated oxidation of 7, rapid intramolecular cyclization would occur to initiate excision and release of free CpG (FIG. 14B). Compound 6 will be synthesized via reaction of NH₂-CpG with isocyanate 8, which will be synthesized by treating Tyr(Bn)-NH—(CH₂)₂—NH-tBoc with triphosgene. The protecting groups in 9 will then be removed to give 6 (FIG. 14C). We will synthesize 7 based on undegradable D-PG polymer to reduce non-specific CpG release. In addition, the corresponding non-targeted CpG conjugate linked through Tyr from PEG-D-PG will also be synthesized as a control.

We will use LC-MS to assess the viability of the tyrosinase-mediated CpG release. Polymer 7 and the nontargeted conjugate will be dissolved in PBS and treated with tyrosinase, and the solution will be analyzed by liquid chromatography-mass spectrometry for evidence of drug release. To ensure that drug release is truly dependent on tyrosinase, the stability of each nanoconstruct in PBS and in bovine serum will also be examined.

### Evaluation of the In Vitro and In Vivo Immunostimulatory Activities of Targeted CpG and Tyrosinase Activatable CpG Nanoconstructs.

Next, we will evaluate the immunostimulatory activity of the tyrosinase-activated nanoconstructs. B16 cells will be treated with each polymer. Twenty four hours later, the culture supernatant will be collected and used for assaying pDC activation using isolated pDC.

### In Vitro Cellular Uptake and Trafficking

Macrophages will be generated from bone marrow of C57BL/6 mice according to our published protocol. To study the internalization of polymer-CpG, macrophages will be pulsed with fluorescent labeled polymers for 1 h, fixed, and stained by monoclonal antibodies toward EEA (early endosome marker), Mannose-6 phosphate receptor (late endosome receptor), and LAMP1 (lysosome marker). Alt antibodies will be from Abcam (Cambridge, Mass.). The colocalization of polymer and different endolysosome markers will be studied by confocal microscopy.

### Cellular Uptake In Vivo

To assess the uptake of CpG-bound PG nanoconstructs in different immune cell populations, each of the three targeted nano-CpG (NDP-MSH-PEG-L-PG-CpG, PEG-D-PG-Tyr-CpG, and DNP-MSH-PEG-D-PG-Tyr-CpG) will be labeled with AlexaFluor 647 and injected intratumorally or intravenously into B16 tumors (n=10). Saline, CpG, non-targeted L-PG-CpG, and non-targeted D-PG-CpG will be used as controls. At 1, 3, and 7 days later, CpG uptake in CD11c+ DC and CD11b+F480+ macrophages will be measured in tumor, draining lymph nodes, and spleen by flow cytometry. In addition, transgenic mice that express GFP in monocyte lineage cells (under control of the murine c-fms promoter) will be used as tumor transplant recipients, which will allow us to study the co-localization of polymer and monocyte-macrophages by using noninvasive imaging in live animals live imaging.

Local cytokine production, delivery to APC in B16 melanoma, activation of pDC, melanoma-specific CD8+ T-cell response in the tumor, and systemic response will be performed as described above.

Antitumor Activity after Intratumoral and Intravenous Injection

The experimental design described in Prophetic Example I, under Validation of Antitumor Activity, will be adapted here for studying antitumor activity of targeted PG-CpG conjugates. Briefly, C57BL/6 mice will be inoculated subcutaneously with B16F10 cells or B16F10 melanoma cells with stable knockdown of tyrosinase geneon both legs (average diameter 4-6 mm). Mice will be assigned to 8 groups (10 mice per group) and treated intratumorally as follows: Group 1, NDP-MSH-PEG-L-PG-CpG targeted to MC1R; Group 2, tyrosinase-activatable DNP-MSH-PEG-D-PG-Tyr-CpG targeted to MC1R; Group 3, tyrosinase-activatable DNP-MSH-PEG-D-PG-Tyr-CpG targeted to MC1R in the treatment of tyrosinase-knockdown tumor; Group 4, no treatment; Group 5, non-targeted PEG-L-PG-CpG; Group 6, tyrosinase-activatable but non-targeted PEG-D-PG-Tyr-CpG; Group 7, tyrosinase-activatable but nontargeted PEG-D-PG-Tyr-CpG in the treatment of tyrosinase-knockdown tumor; Group 8, non-degradable, non-targeted nanoconstruct PEG-D-PG-CpG. Groups 4-8 serve as controls. For all groups except groups 3 and 7, B16F10 cells will be used. For groups 3 and 7, B16F10 cells with stable knockdown of tyrosinase gene by RNA interference will be used. The stable knockdown of tyrosinase gene will be performed by stable transfection of commercially available plasmid from Invitrogen (Carlsberg, Calif.). Mice in each group will receive intratumoral injection of each agent on days 1, 7, and 10 at a dose of 50 μg equivalent CpG/injection (100 μl). Tumor size will be measured on day 21, and the animals will be sacrificed and draining lymph nodes, spleen, and both treated and untreated tumors will be removed for IHC and FACS analysis of pDC population and cell death (TUNEL). Weights of individual tumors will be recorded and used as a measure of tumor control on day 21.

In a separate study, tumor-bearing mice will be divided into 8 groups consisting of 10 mice in each group. Mice in each group will be injected intravenously with the same agent as outlined above, and antitumor activity determined as described above. Statistical analysis will be performed as described in Example I, under Data Analysis and Statistics.

### Anticipated Results, Pitfalls and Solutions

We expect highly selective activation of tumor-specific immune response with both MCR1-targeted nanoconstructs and tyrosinase-activatable nanoconstructs. However, because tyrosinase is unique to melanoma, it is anticipated that the tyrosinase-activatable nanoconstructs will induce a more specific response than the targeted nanoconstructs.

In the unlikely event that tyrosinase-mediated CpG release from nanoconstructs is not successful, we will replace tyrosine with L-DOPA as the tyrosinase substrate. Alternatively, a carbamate instead of urea linker may be used. These approaches are expected to be effective in mediating release of CpG. The main pitfall in this Prophetic Example is that even after our intensive efforts, MC1R targeted and tyrosinase-mediated CpG release may not be able to avoid stimulation of immune cells systemically. Losing the “focus” of activating melanoma-specific immune response would adversely affect the effectiveness of CpG therapy. In the event that such a scenario occurs, we will pursue two alternative approaches. First, we will attempt targeting L-PG-CpG to other melanoma-specific biomarkers. We will use monoclonal antibodies specific for melanoma cells, such as anti-GD2 antibody and anti-GM3 antibody as the homing ligands. These antibodies have high affinity (Kd=10 nM) to the cell surface receptors and may improve the binding of nano-CpG to tumor cells. Second, we will use tong-circulating core-crosslinked polymeric micelles as the carriers of CpG. We have previously shown that these nanomaterials can efficiently evade the cells of monophagocytic system. By preventing the uptake of nano-CpG by monophagocytic system, we expect to reduce the systemic uptake and release of nano-CpG outside of tumor.

### Prophetic Example III

### The Effect of L-PG-CpG Nanoconstructs Used Alone and in Combination with T Regulatory Cell Depletion on Antitumor Immunity

The combination of intratumoral injection of nano-CpG (best from Example I) or intravenous injection of targeted nano-CpG (best from Example II) and therapies targeting costimulatory pathways will lead to robust antitumor activity through activation of multiple innate and adaptive immune cells. Additionally, these treatment plans can further incorporate the use of such cytokines as IL-2 and IFN-α, which have shown some promise in the clinical area, but still have room for improvement.

### Rational and Overall Strategy

It is generally accepted that effective immunotherapy of cancer depend on acting on multiple checkpoints of the immune stimulation. Combined use of immunotherapeutic agents that synergize through different mechanisms has been a critical area of research. Full activation of immunity requires stimulation of positive costimulatory signals and inhibition of negative immune regulatory signals. Successful immunotherapy will involve the rational combination of agents to activate the critical steps involved in the development of a strong immune response.

Therefore, after having determined the most effective antitumor nano-CpG in the B16 melanoma murine model, we will combine these TLR agonists with systemic immunomodulation, either with positive immunostimulatory agent, such as angonist antibodies against OX40, CD40, and 4-1BB, or with negative costimulatory molecules, such as antibodies against B7 family molecules B7S1, and B7H3, and anti-CTLA-4. Once the optimal combination is identified, it can be used to build upon cytokine therapy with IL-2 and IFN-α, given previous moderate clinical success with these cytokines.

Each of these agonist antibodies to costimulatory molecules has been evaluated in conjunction with a vaccine to a TAA. Combining these antibodies with a TLR agonist in lieu of a vaccine has the potential to vaccinate the patient against multiple tumor-specific antigens, tailored to an individual patient's tumor. Lastly, the moderate clinical success seen with the administration of IL-2 and IFN-α to melanoma patients leaves room for improvement, potentially through the addition of a TLR-agonist antibody combination.

### Research Plan

Evaluation of Effective Nano-CpG Treatment with Either Agonist Antibodies to Costimulatory Molecules or B7 Negative Costimulatory Molecules

Mice will be implanted with B16 melanoma. One week later, after solid tumors have been established, treatment will be started with either of the two TLR agonists (from the best candidates from Examples 1 and 2 above) plus either agonist antibodies to OX40, CD40, or 4-1BB or antagonist antibodies toward B7 negative costimulatory molecules (anti-B7S1 and anti-B7H3 antibodies) and anti-CTLA-4, all given intraperitoneally. The route of TLR agonist administration will depend on the agonist determined to be the most effective. Antibody will be administered 5 days after nano-CpG injection at a predetermined dose.

Our preliminary data indicate that tumor-specific CD8 T cell response peaked at 5 days after intratumoral injection of L-PG-CpG (FIG. 5). Endpoints will be tumor size reduction and mouse survival. The mechanisms underlying the optimal synergistic combinations will also be investigated, with specific regard to cellular changes within the tumor microenvironment, development of antigen-specific T cells, and CTL activity, and further modification will be studied based on the findings. We expect that the combination of TLR agonists with antibody mediated engagement of stimulatory pathways will further boost the antitumor response

Evaluation of Effective Nano-CpG and Either Agonist Antibodies to Costimulatory Pathways or B7 Negative Costimulatory Molecules in Conjunction with Cytokine Regimens

Mice will be implanted with B16 melanoma, and treatment will be started 1 week later. Having identified an optimal combination of TLR agonist plus stimulatory antibody or B7 negative costimulatory molecule, IL-2 and IFN-α will be added to this regimen. IL-2 or IIFN-α will be given intraperitoneally together with antibodies.

### Monitoring Therapeutic Response of Combination Therapy

### Treatment Regiments

The following groups will be studied for the best nano-CpG identified from Prophetic Example I:

Combination with antagonist antibodies toward negative costimulatory pathways: Group 1, nano-CpG; Group 2, nano-CpG+anti-CTLA-4; Group 3, anti-B7S1+nano-CpG; Group 4, anti-B7H3+nano-CpG.

Combination with agonist antibodies toward positive stimulatory pathways: Group 5, nano-CpG+anti-OX40; Group 6, nano-CpG+anti-CD40; Group 7, nano-CpG+anti-4-1-1BBL.

Combination with cytokines: Group 8, nano-CpG IL-2+on costimulatory pathway reagent; Group 9, nano-CpG+IFN-α+one costimulatory pathway reagent.

The same treatment plan will be instituted for the best nano-CpG identified in Prophetic Example II above.

Data Analysis and Statistics: Statistical analysis will be performed as described in Example I, under Data Analysis and Statistics. Local cytokine production, delivery to APC in B16 melanoma, activation of pDC, melanoma-specific CD8+ T-cell response in the tumor, and systemic response will be performed as described above.

### Anticipated Results, Pitfalls and Solutions

We expect to observe enhanced antitumor response by combined use of costimulatory reagents and cytokines. Especially, we expect to see much improved effect on treatment of distal tumors due to the enhanced tumoricidal activity of CD8 T cells caused by costimulatory reagents.

Toxicity of immunomodulators as observed for IFN-α and IL-2 may prevent their combined use with nano-CpG. Since nano-CpG has been designed to avoid systemic activation of the immune system, it is unlikely that nano-CpG will enhance the known toxicity of costimulatory reagents and cytokines. As an alternative approach, we have two plans: 1) we will reduce the dose of the single reagent as we used in the preliminary studies and examine whether nano-CpG reduces the required effective dose for that specific reagent; and/or 2) we will try low doses of multiple costimulatory reagents to examine whether synergism among these reagents reduces the required dose for each individual reagent.

### Prophetic Example IV

### In Vivo Analysis

Female mice (about 600 per year) of C57BL/6 inbred strain and GFP-transgenic mice will be used for these experiments.

The major procedures to be performed with mice include the following:
Since in vivo tumor elimination and in vivo immune responses are keys to understanding the anticancer efficacy of the nanocarrier-CpG candidates being developed, the proposed studies can only be tested in animals. For statistical significance of the data generated, we will repeat the assay at least once and use 10 mice per test per group.
The animals will be maintained in a pathogen-free holding facility for small animal, at the M. D. Anderson where alternating 12-h periods of light and darkness, temperature, and humidity are controlled as approved by the American Association for the Accreditation of Laboratory Animal Care (AAALAC). All procedures will be performed by trained staff and approved by the Institutional Animal Care and Use Committee.
Mice will be anesthetized with a ketamine/xylazine mixture equivalent to 10 mg/mL ketamine and 1 mg/mL xylazine delivered I.P. The anesthesia reagents to be used are standard and found to be safe and approved for use in mice. During immunizations under anesthesia, the mice will be observed for any problems and during the entire period they will be kept warm.

The tumor inoculation will be performed by injection of 3×10⁵ B16 melanoma cells on left and/or right flank of mice. The intratumoral injection of nano-CpG and controls will be performed 7 days after tumor inoculation. The manipulations of animals inoculated by adenoviral vector transduced cell lines will be under BSL-2 conditions in the animal facility with BSL-2 practices for the personnel performing the experiments.
Tumor growth in mice will be measured 2-3 times a week. Mice will be sacrificed when tumor size reach 1.2 cm in diameter (about 21 days after 3×10⁵ B16 cells inoculation). The anticancer effect of nano-CpG will be studied during the 21 days period.
Animals will be inspected daily for well-being and any animals that become moribund during the course of the study will be euthanized. Morbidity will be determined on the basis of the animal's physical appearance, activity level, appetite, and respiratory rate.
The blood samples will be drawn in anesthetized animals. At the end of experiment, the animals will be sacrificed and various tissues (spleen, lymph nodes, and tumor) will be harvested for T cell assays, and serum for cytokine assays.
In all these procedures no toxic events are expected, since the nano-CpG and CpG are not toxic, and recombinant viruses used to transduce B16 melanoma cells are replication defective. The doses to be used are all lower than those known to be toxic in published studies. Any animal showing lethargy, ruffled fur, or distress will be sacrificed to avoid prolonged distress. Mice will be immediately sacrificed if ulcer is observed in tumor.
Euthanasia will involve CO₂ exposure. Once the animals are asleep and non-responsive to external stimuli, they will be sacrificed by cervical dislocation. This process is consistent with the recommendations of the Panel on Euthanasia of the AVMA. Following tissue harvest, the animal carcasses will be disposed of in the bio-hazardous animal waste.

The following Tables include the various groups of mice used in the different experiments proposed above (Prophetic Examples I, II and II), and time line.

TABLE 2
For Prophetic Example I
Total: 620 C57BL/6 mice, 120 transgenic mice.

| | | | Time Study |
|---|---|---|---|
| Group | Treatment with | Route | Initiated |
| 1 | Saline control | Intratumoral | Year 1-2 |
| 2-12 | 11 types of Nano-CpG | Intratumoral | Year 1-2 |
| 13 | Free CpG | Intratumoral | Year 1-2 |

### Prophetic Example I Validation of Antitumor Activity

Tumor retention: 10 mice/group×13 groups=130 mice

### Prophetic Example I

### Evaluation of the Innate and Acquired Immunity Induced by CpG and PG-Based Nanocontructs after Intratumoral Injection

pDC uptake in vivo, 10 mice/group×groups (excluding saline)×3 time points=360 mice
transgenic mice. 10 mice/group×12 groups=120 transgenic mice

TABLE 3
For Prophetic Example II, Total: 300C57BL/6
mice, 140 transgenic mice.

| | | | Time Study |
|---|---|---|---|
| Group | Treatment with | Route | Initiated |
| 1 | NDP-MSH-PEG-L-PG-CpG | Intratumoral | Year 2-3 |
| 2 | Tyrosinase-activatable | Intratumoral | Year 2-3 |
| | DNP-MSH-PEG-D-PG-Tyr-CpG | | |
| | targeted to MC1R | | |
| 3 | Tyrosinase-activatable | Intratumoral | Year 2-3 |
| | DNP-MSH-PEG-D-PG-Tyr-CpG | | |
| | targeted to MC1R in the | | |
| | treatment of tyrosinase | | |
| | knockdown tumor | | |
| 4 | Non-targeted PEG-L-PG-CpG | Intratumoral | Year 2-3 |
| 5 | Tyrosinase-activatable but | Intratumoral | Year 2-3 |
| | non-targeted PEG-D-PG-Tyr-CpG | | |
| 6 | Tyrosinase-activatable but | Intratumoral | Year 2-3 |
| | non-targeted PEG-D-PG-Tyr-CpG | | |
| 7 | Non-degradable, non-targeted | Intratumoral | Year 2-3 |
| | nanoconstruct PEG-D-PG-CpG | | |
| 8 | PBS control | Intratumoral | Year 2-3 |
| 9-16 | Groups 1-8 | Intravenous | Year 3-4 |

### Prophetic Example II

### Evaluation of the In Vitro and In Vivo Immunostimulatory Activities of Targeted CpG and Tyrosinase Activatable CpG Nanoconstructs

Uptake in pDC: 10 mice/group×7 groups (excluding tyrosinase knockdown tumor)×2 injection routes=140 mice
GFP transgenic mice: 10 mice/group×7 groups×2 injection routes=140 transgenic mice
Prophetic Example II, Antitumor activity: 10 mice/group×16 groups=160 mice.

TABLE 3a
For Prophetic Example III
Nano-CpG (best from Example I) in
combination with antibodies, total 210 mice.

| | | | Time Study |
|---|---|---|---|
| Group | Treatments | Route | Initiated |
| 1 | Nano-CpG | Intratumoral | Year 3 |
| 2 | Nano-CpG + anti-CTLA4 | Intratumoral | Year 3 |
| 3 | Nano-CpG + anti-B7S1 | Intratumoral | Year 3 |
| 4 | Nano-CpG + antiB7H3 | Intratumoral | Year 3 |
| 5 | Nano-CpG + anti-OX40 | Intratumoral | Year 3 |
| 6 | Nano-CpG + anti-CD40 | Intratumoral | Year 3 |
| 7 | Nano-CpG + anti-4-1BB | Intratumoral | Year 3 |
| 8 | Nano-CpG + IL2 | Intratumoral | Year 4 |
| 9 | Nano-CpG + anti-CTLA4 + IL2 | Intratumoral | Year 4 |
| 10 | Nano-CpG + anti-B7S1 + IL2 | Intratumoral | Year 4 |
| 11 | Nano-CpG + antiB7H3 + IL2 | Intratumoral | Year 4 |
| 12 | Nano-CpG + anti-OX40 + IL2 | Intratumoral | Year 4 |
| 13 | Nano-CpG + anti-CD40 + IL2 | Intratumoral | Year 4 |
| 14 | Nano-CpG + anti-4-1BB + IL2 | Intratumoral | Year 4 |
| 15 | Nano-CpG + IFNa | Intratumoral | Year 4 |
| 16 | Nano-CpG + anti-CTLA4 + IFNa | Intratumoral | Year 4 |
| 17 | Nano-CpG + anti-B7S1 + IFNa | Intratumoral | Year 4 |
| 18 | Nano-CpG + antiB7H3 + IFNa | Intratumoral | Year 4 |
| 19 | Nano-CpG + anti-OX40 + IFNa | Intratumoral | Year 4 |
| 20 | Nano-CpG + anti-CD40 + IFNa | Intratumoral | Year 4 |
| 21 | Nano-CpG + anti-4-1BB + IFNa | Intratumoral | Year 4 |

TABLE 3b
Prophetic Example III, Antitumor activity:
10 mice/group × 21 groups = 210 mice.
Nano-CpG (the best from Prophetic Example II)
in combination with antibodies, total 210 mice,

| | | | Time Study |
|---|---|---|---|
| Group | Treatment with | Route | Initiated |
| 1 | Nano-CpG | Intratumoral | Year 4 |
| 2 | Nano-CpG + anti-CTLA4 | Intratumoral | Year 4 |
| 3 | Nano-CpG + anti-B7S1 | Intratumoral | Year 4 |
| 4 | Nano-CpG + antiB7H3 | Intratumoral | Year 4 |
| 5 | Nano-CpG + anti-OX40 | Intratumoral | Year 4 |
| 6 | Nano-CpG + anti-CD40 | Intratumoral | Year 4 |
| 7 | Nano-CpG + anti-4-1BB | Intratumoral | Year 4 |
| 8 | Nano-CpG + IL2 | Intratumoral | Year 5 |
| 9 | Nano-CpG + anti-CTLA4 + IL2 | Intratumoral | Year 5 |
| 10 | Nano-CpG + anti-B7S1 + IL2 | Intratumoral | Year 5 |
| 11 | Nano-CpG + antiB7H3 + IL2 | Intratumoral | Year 5 |
| 12 | Nano-CpG + anti-OX40 + IL2 | Intratumoral | Year 5 |
| 13 | Nano-CpG + anti-CD40 + IL2 | Intratumoral | Year 5 |
| 14 | Nano-CpG + anti-4-1BB + IL2 | Intratumoral | Year 5 |
| 15 | Nano-CpG + IFNa | Intratumoral | Year 5 |
| 16 | Nano-CpG + anti-CTLA4 + IFNa | Intratumoral | Year 5 |
| 17 | Nano-CpG + anti-B7S1 + IFNa | Intratumoral | Year 5 |
| 18 | Nano-CpG + antiB7H3 + IFNa | Intratumoral | Year 5 |
| 19 | Nano-CpG + anti-OX40 + IFNa | Intratumoral | Year 5 |
| 20 | Nano-CpG + anti-CD40 + IFNa | Intratumoral | Year 5 |
| 21 | Nano-CpG + anti-4-1BB + IFNa | Intratumoral | Year 5 |

Each experiment will be repeated 2-3 times. Total Estimated Mice: ˜3760 mice.

What is claimed is:

1. A nanoconstruct used to treat melanoma comprising poly(L-glutamic acid)-CpG conjugate, wherein said nanoconstruct provides targeted delivery of CpG to melanoma in vivo and enhancing melanoma antitumor activity while reducing or eliminating systemic activation of pDC.

2. A delivery system for administering an immunomodulator to a tumor site comprising nanoconstruct wherein a nanopolymer is conjugated to an immunomodulatory wherein said immunomodulatory is CpG, or a combination of CpG and other tumor binding ligands and antibodies.

3. The delivery system of claim 2, wherein the nanopolymer is poly(L-glutamic acid) and the other tumor ligands are MSH derived ligands for melanocortin type 1 receptor (MC1R), apatamers, or antibodies.

4. A method of treating melanoma to a subject in need thereof comprising the step of administering to the subject a therapeutic amount of a nanoconstruct having CpG conjugated to an macrophage-tropic polymer.

5. A method of treating melanoma to a subject in need thereof comprising the step of administrating to the subject a therapeutic amount of nanopolymer-conjugated immunodrugs.

6. The method of claim 5, wherein the nanopolymer-conjugated immunodrug is poly(L-glutamic acid)-CpG conjugate (“L-PG-Cp-G”).

7. The method of claim 5, wherein agonists of positive costimulatory signals and inhibitors of negative immune regulatory signals are co-administered to the subject.
